(19) United States
(12) Patent Application Publication
(10) Pub. No.: US 2022/0024840 A1
(43) Pub. Date: Jan. 27, 2022

(54) SELECTIVE TERMINAL FUNCTIONALIZATION OF ALKANES

(71) Applicant: TECHNION RESEARCH & DEVELOPMENT FOUNDATION LIMITED, Haifa (IL)

(72) Inventors: Ilan MAREK, Haifa (IL); Yechezkel KASHI, Hayogev (IL); Jeffrey BRUFFAERTS, Akko (IL); Keren BUHNIK -ROSENBLAU, Kibbutz Usha (IL); Morgan CORMIER, Haifa (IL); Guy HOREV, Kiryat Tivon (IL); Inbar KESTEN, Haifa (IL); Anthony COHEN, Haifa (IL)

(21) Appl. No.: 17/297,615

(22) PCT Filed: Nov. 28, 2019
(86) PCT No.: PCT/IL2019/051310

§ 371 Date: May 27, 2021

Related U.S. Application Data
(60) Provisional application No. 62/772,203, filed on Nov. 28, 2018.

(51) Int. Cl. C07C 17/08; C07C 51/373; C07C 5/333; C07C 209/02; C07C 253/30; C07F 7/08; C07F 5/02; C12P 5/02

(52) U.S. Cl. CPC C07C 17/08; C07C 51/373; C07C 5/3335; C07C 209/02; C12P 5/026; C07F 7/0805; C07F 7/081; C07F 5/027; C07C 253/30

(57) ABSTRACT

The present invention provides a method for selectively functionalizing alkanes through a sequential biocatalytic dehydrogenation followed by isomerization-hydrofunctionalization reaction.

### CROSS REFERENCE TO RELATED APPLICATIONS

This application claims the benefit of priority to U.S. Provisional Patent Application No. 62/772,203 filed Nov. 28, 2018, entitled “SELECTIVE TERMINAL FUNCTIONALIZATION OF ALKANES”, the contents of which is incorporated herein by reference in its entirety.

### FIELD OF THE INVENTION

The present invention, in some embodiments thereof, relates to biocatalysis and organometallic chemistry and their use for selective functionalization of alkanes.

### BACKGROUND

Alkanes are major occurring compounds found in crude oil and other natural resources. Generally employed as fuels, their use in synthesis has particularly been limited due to their inertness. Most existing alkane activation systems rely on harsh conditions, henceforth drastically hampering selectivity.

Previous reported alkane chemical activation methods generally describe low-yielding or poorly selective transformations.

To date, there is no general solution for the efficient selective activation of alkanes longer than C5. The existing chemical methods use an excess of the alkane and therefore are less atom-economical. Moreover, the only existing methods are usually highly energy-consuming processes (high temperatures and pressures, e.g. alkane cracking) or using expensive noble transition metal catalysts (e.g. rhodium or iridium).

### SUMMARY

According to one aspect, there is provided a method for selectively functionalizing alkanes, comprising: (a) providing an alkane; (b) dehydrogenating at least one saturated hydrocarbon of the alkane by a hydrocarbon-utilizing microorganism; followed by (c) isomerization-hydrofunctionalization reaction; thereby yielding a functionalized alkane or derivative thereof.

In some embodiments, the alkane comprises at least one terminal C—H bond, wherein the alkane is selectively functionalized at one of the terminal C—H bonds at a yield of at least 98%.

In some embodiments, the hydrocarbon-utilizing microorganism is selected from the group consisting of a yeast, a cyanobacterium, and a bacterium.

In some embodiments, the hydrocarbon-utilizing microorganism is characterized by having overexpression of at least one gene selected from the group consisting of: WP_003945297.1; WP_065352723.1; WP_065352722.1; WP_065352721.1; WP_050656813.1; WP_007726140.1; WP_007726141.1; WP_065351823.1; WP_003941324.1; WP_019749215.1; WP_003941638.1; WP_042450649.1; WP_003941322.1; WP_065351660.1; WP_003945295.1; WP_042450854.1; WP_065351492.1; WP_003940783.1; WP_065351661.1; WP_065352563.1; WP_065351754.1; WP_030536487.1; WP_065351402.1; WP_080726744.1; WP_042449699.1; WP_065351224.1; WP_003941275.1; WP_003939932.1; WP_007729732.1; WP_003946304.1; WP_042452232.1; WP_007726566.1; WP_054187331.1; WP_003941308.1; WP_007726568.1; WP_058227688.1; WP_065351782.1; WP_065352072.1; WP_020968150.1; WP_003941552.1; WP_065352440.1; WP_003942314.1; WP_042450108.1; WP_007735391.1; WP_065352885.1; WP_007727244.1; WP_065352214.1; and WP_003943732.1, compared to a control.

In some embodiments, the hydrocarbon-utilizing bacterium is Rhodococcus mutant strain KSM-B-3M.

In some embodiments, the alkane is a linear, branched or cyclic alkane.

In some embodiments, the alkane is a C4 to C40 alkane.

In some embodiments, the alkane further comprises a functional group, an aryl substituent, or a combination thereof.

In some embodiments, the functionalized alkane or derivative thereof has a general formula RCₙH₂ₙR¹, wherein n is an integer having a value of 4 to 40, and wherein R, R¹ are each independently selected from a hydrogen atom, a halogen atom, a nitro group, an amine group, an azido group, a cyano group, a methoxy group, a carboxylic group, an ester group, an ether group, an aromatic group, alkyl group, vinyl group, an alcohol group, a carbamate group, an urea group or a combination thereof.

In some embodiments, the alkane is a C13 to C20 alkane.

In some embodiments, the functionalized alkane or derivative thereof has a general formula RCₙH₂ₙR¹, wherein n has a value of 13 to 20.

In some embodiments, the yield of step (b) is at least 18%.

In some embodiments, step (b) is performed in an aqueous medium selected from the group consisting of phosphate buffer, an amino acid, thiamine hydrochloride and magnesium sulfate heptahydrate, or any combination thereof.

In some embodiments, step (b) is performed at a temperature ranging from 20° C. to 34° C.

In some embodiments, the pH of the aqueous medium is within the range of 6.0 to 6.8.

In some embodiments, step (b) is performed for at least 5 days.

In some embodiments, step (c) is metal assisted isomerization-hydrofunctionalization reaction.

In some embodiments, the isomerization-hydrofunctionalization reaction is selected from the group of halogenolysis, oxidation, copper-catalyzed allylation, hydroboration, hydrosilylation, hydrozirconation, hydroarylation and hydroamination.

In some embodiments, step (c) functionalizes the alkane with a terminal covalent bond selected from: C—C, C—O, C—X, wherein X is halogen, Si, N, B or any combination thereof.

In some embodiments, step (b) further comprises: (i) recovering the microorganism; and (ii) re-dissolving the microorganism in aqueous medium and performing another dehydrogenation reaction.

According to one aspect, there is provided a composition comprising at least two cis-alkene regioisomers and an alkane wherein the ratio of the at least two cis-alkenes to the alkane is in a range of 95:5 to 80:20.

In some embodiments, the alkane is hexadecane and the cis-alkene regioisomers are about 80% cis-7-hexadecene and about 20% cis-8-hexadecene, and wherein the ratio of the at least two cis-alkenes to the alkane is in a range of 90:10 to 80:20.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 presents a phylogram visualization of the phylogenetic analyses of Rhodococcus strains.

FIG. 2 presents the gas chromatography (GC) chromatogram for cis-hexadecenes (1).

FIG. 3 presents the GC chromatogram for cis-tetradecenes (“2”).

FIG. 4 presents the GC chromatogram for cis-pentadecenes (“3”).

FIG. 5 presents the GC chromatogram for cis-heptadecenes (“4”).

FIG. 6 presents the GC chromatogram for cis-octadecenes (“5”).

FIG. 7 presents the GC chromatogram for cis-eicosenes (“6”).

FIG. 8 presents the GC chromatogram for cis-1,n-hexadecadienes (“7”).

FIG. 9 presents the GC chromatogram for cis-1,n-octadecadienes (“8”).

FIG. 10 presents the GC chromatogram for 1-methoxy-cis-hexadecenes (“9”).

FIG. 11 presents the GC chromatogram for 1-chloro-cis-hexadecenes (“10”).

FIG. 12 presents the GC chromatogram for 1-chloro-cis-octadecenes (“11”).

FIG. 13 presents the GC chromatogram for 1-fluoro-cis-hexadecenes (“12”).

FIG. 14 presents the GC chromatogram for 1-fluoro-cis-octadecenes (“13”).

FIG. 15 presents the chemical structure and name of the compounds with low productivity in the dehydrogenation reaction.

FIG. 16 presents the chemical structure and name of the compounds with no productivity in the dehydrogenation reaction.

FIG. 17 presents the results of dehydrogenation reaction performed with isotopically labelled hexadecane.

FIG. 18 presents the GC chromatogram for 1-iodohexadecane as a mixture with remaining hexadecane.

FIG. 19 presents the GC chromatogram for 1-bromohexadecane as a mixture with remaining hexadecane.

FIG. 20 presents the GC chromatogram for 1-chlorohexadecane as a mixture with remaining hexadecane.

FIG. 21 presents the GC chromatogram for 1-nonadecene as a mixture with hexadecane.

FIG. 22 presents the GC chromatogram for nonadec-1,2-diene as a mixture with hexadecane.

FIG. 23 presents the GC chromatogram for hexadecyltrimethylsilane as a mixture with hexadecane.

FIG. 24 presents the biphasic suspension obtained when recycling Rhodococcus mutant strain KSM-3-BM.

FIG. 25 presents the recycled Rhodococcus mutant strain KSM-3-BM as an orange pellet.

FIG. 26 presents a bar graph showing the reaction conversion of the recycling experiments of Rhodococcus mutant strain KSM-3-BM.

FIGS. 27A-27Y present NMR chromatograms of compounds 1-13.

FIGS. 28A-27Y present NMR chromatograms of compounds 14-25.

FIGS. 29A-27H present NMR chromatograms of compounds 26-29.

FIGS. 30A-30B is a vertical bar graphs showing biomass accumulation (30A) and organic phase (30B) leftovers obtained by strains KSM-3BM and sp. 008 along a 21 days experiment.

FIG. 31 is a graph showing principal component analysis of RNAseq patterns in strains KSK-3BM and sp. 008 on hexadecane and dodecane.

FIGS. 32A-32B are volcano plots representing the significance of each gene vs its expression difference between strains. Genes that show extreme differences are indicated. (32A) shows genes that are overexpressed in KSM-3BM by at least ×2. (32B) shows the genes overexpressed in sp. 008 by at least ×2. 1—acyl-CoA desaturase; 2—PTS N-acetylglucosamine transporter subunit IIBC; 3—phosphoenolpyruvate protein phosphotransferase; 4—N-acetylglucosamine-6-phosphate deacetylase; 5—ferredoxin reductase; 6—glucosamine-6-phosphate deaminase; 7—copper homeostasis protein CutC; 8—HNH endonuclease; 9—PTS sugar transporter; 10—NDMA-dependent alcohol dehydrogenase; 11—PTS glucose transporter subunit IIA; 12—GTP-binding protein; 13—HPr family phosphocarrier protein; 14—nitrile hydratase subunit beta; 15—hypothetical protein; and 16—nitrile hydratase subunit alpha.

FIG. 33 is an illustration showing the silencing of the beta oxidation pathway in KSM 3BM as observed by the DAVID database based on a KEGG pathway map. Stars represent proteins detected at significantly lower abundance in the KSM 3BM proteome.

### DETAILED DESCRIPTION

According to some embodiments, the present invention provides a method for functionalizing alkanes, which comprises dehydrogenating at least one saturated hydrocarbon by a hydrocarbon-utilizing microorganism; followed by isomerization-hydrofunctionalization reaction.

According to some embodiments, the method comprises: (a) providing an alkene obtained by dehydrogenation of at least one saturated hydrocarbon of an alkane using a hydrocarbon-utilizing microorganism; followed by (b) isomerization-hydrofunctionalization reaction; thereby yielding a functionalized alkane or derivative thereof.

According to one aspect, the alkane comprises a terminal C—H bond, wherein the selectivity to functionalize the alkane at the terminal C—H bond compared to a non-terminal C—H bond is 98% or more.

By the term “terminal bond” it is meant a bond at the terminal end of chain or of a substituent.

In some embodiments, the selectivity to functionalize the alkane at the terminal C—H bond is 98%. In some embodiments, the selectivity to functionalize the alkane at the terminal C—H bond is about 98.5%, about 99%, about 99.5%, or about 99.9%, including any value therebetween.

In some embodiments, the method provides functionalized alkanes with an overall yield that ranges from 5% to 60%. In some embodiments, the method provides functionalized alkanes with an overall yield of 5%, 10%, 15%, 20%, 25%, 30%, 35%, 40%, 45%, 50%, 55%, or 60%, including any value and range therebetween.

As used herein, the term “alkane” means saturated hydrocarbons or compounds that comprises carbon (C) and hydrogen (H), wherein the carbon atoms are linked together by single bonds (i.e., they are saturated compounds). The term “alkane”, as used herein, also encompasses a hydrocarbon chain having 1, 2, or 3, unsaturated hydrocarbon, and also encompasses substituted alkane.

In some embodiments, the alkane is selected from a linear chain alkane, branched chain alkane or cycloalkane. In some embodiments, the branched chain alkane may have one or more points of branching. In some embodiments the branched chain alkane may include cyclic branches.

In some embodiments, the alkane further comprises any one of functional groups, aryl substituents or a combination thereof.

In some embodiments, the alkane has a general formula RCₙH₂ₙR¹, in which n is an integer having a value of at least 3. In some embodiments n=4 to 40 carbons in length, and R, R¹ are each independently selected from a hydrogen atom, a halogen group, a nitro group, an amine group, a methoxy group, a carboxylic group, an ester group, an aromatic group, alkyl group, vinyl group, alcohol group, ether group, azido group, cyano group, carbamate group, urea group or a combination thereof. In some embodiments, the alkane has a general formula RCₙH₂ₙR¹, in which n=14 to 18 carbons in length, and R, R¹ are each independently selected from a hydrogen atom, a halogen group, a nitro group, an amine group, a methoxy group, a carboxylic group, an ester group, an aromatic group, alkyl group, vinyl group, alcohol group, ether group, azido group, cyano group, carbamate group, urea group or a combination thereof. In some embodiments, the alkane has a general formula RCₙH₂ₙR¹, in which n=4 to 40 carbons in length, and R, R¹ are each independently selected from a hydrogen atom, a halogen group, a nitro group, an amine group, a methoxy group, a carboxylic group, an ester group, an aromatic group, alkyl group, vinyl group, alcohol group, ether group, azido group, cyano group, carbamate group, urea group or a combination thereof.

In some embodiments, the alkane has a general formula RCₙH₂ₙR¹, in which n is an integer having a value of at least 3. In some embodiments n=4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 carbons in length, and R, R¹ are each independently selected from a hydrogen atom, a halogen group, a nitro group, an amine group, a methoxy group, a carboxylic group, an ester group, an aromatic group, alkyl group, vinyl group, alcohol group, ether group, azido group, cyano group, cyclic group, carbamate group, urea group or a combination thereof.

In some embodiments, the alkane has a general formula RCₙH₂ₙR¹, in which R, R¹ can be saturated or unsaturated. In some embodiments, the alkane has a general formula RCₙH₂ₙR¹, in which R, R¹ can have one or more points of unsaturation.

In some embodiments, the functionalized alkane has a general formula RCₙH₂ₙR¹, in which n is an integer having a value of at least 3. In some embodiments n=4 to 40 carbons in length, and R, R¹ are each independently selected from a hydrogen atom, a halogen group, a nitro group, an amine group, a methoxy group, a carboxylic group, an ester group, an aromatic group, alkyl group, vinyl group, alcohol group, ether group, azido group, cyano group, carbamate group, urea group or a combination thereof. In some embodiments, the alkane has a general formula RCₙH₂ₙR¹, in which n=14 to 18 carbons in length, and R, R¹ are each independently selected from a hydrogen atom, a halogen group, a nitro group, an amine group, a methoxy group, a carboxylic group, an ester group, an aromatic group, alkyl group, vinyl group, alcohol group, ether group, azido group, cyano group, carbamate group, urea group or a combination thereof. In some embodiments, the functionalized alkane has a general formula RCₙH₂ₙR¹, in which n=4 to 40 carbons in length, and R, R¹ are each independently selected from a hydrogen atom, a halogen group, a nitro group, an amine group, a methoxy group, a carboxylic group, an ester group, an aromatic group, alkyl group, vinyl group, alcohol group, ether group, azido group, cyano group, carbamate group, urea group or a combination thereof.

In some embodiments, the functionalized alkane has a general formula RCₙH₂ₙR¹, in which n is an integer having a value of at least 3. In some embodiments n=4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 carbons in length, and R, R¹ are each independently selected from a hydrogen atom, a halogen group, a nitro group, an amine group, a methoxy group, a carboxylic group, an ester group, an aromatic group, alkyl group, vinyl group, alcohol group, ether group, azido group, cyano group, cyclic group, carbamate group, urea group or a combination thereof.

In some embodiments, the functionalized alkane has a general formula RCₙH₂ₙR¹, in which R, R¹ can be saturated or unsaturated. In some embodiments, the alkane has a general formula RCₙH₂ₙR¹, in which R, R¹ can have one or more points of unsaturation.

By the term “independently”, as used herein, it is meant that the R and R¹ substituents may or may not be identical.

The term “non-functionalized” refers to alkanes having a sp3 hybridized terminal CH₃ group. In contrast, the terms “functionalize,” “functionalized” “functionalizing” and “functionalization” refer to alkanes, wherein one hydrogen of the sp hybridized terminal CH₃ group is replaced by a different atom or group.

As used herein, the terms “halo” and “halogen” refers to a substituent which may be fluoro, chloro, bromo, or iodo.

In some embodiments, the functionalized alkanes are derived from linear, branched, cyclic or acyclic alkanes having from 4 to 40 carbon atoms, which in each case form the parent structure.

As used herein, the term “alkene” refers to linear, branched, cyclic or acyclic hydrocarbon having the number of carbon atoms indicated in the prefix and containing at least one double bond.

### Microorganism

In some embodiments, the hydrocarbon-utilizing microorganism is selected from a yeast, a fungi, a filamentous fungi, a cyanobacteria, an algae, and a bacteria.

In some embodiments, the hydrocarbon-utilizing microorganism is characterized by having overexpression of at least one gene involved in fatty acid metabolism, compared to control.

In some embodiments, the hydrocarbon-utilizing microorganism is characterized by having overexpression of at least one gene selected from: WP_003945297.1; WP_065352723.1; WP_065352722.1; WP_065352721.1; WP_050656813.1; WP_007726140.1; WP_007726141.1; WP_065351823.1; WP_003941324.1; WP_019749215.1; WP_003941638.1; WP_042450649.1; WP_003941322.1; WP_065351660.1; WP_003945295.1; WP_042450854.1; WP_065351492.1; WP_003940783.1; WP_065351661.1; WP_065352563.1; WP_065351754.1; WP_030536487.1; WP_065351402.1; WP_080726744.1; WP_042449699.1; WP_065351224.1; WP_003941275.1; WP_003939932.1; WP_007729732.1; WP_003946304.1; WP_042452232.1; WP_007726566.1; WP_054187331.1; WP_003941308.1; WP_007726568.1; WP_058227688.1; WP_065351782.1; WP_065352072.1; WP_020968150.1; WP_003941552.1; WP_065352440.1; WP_003942314.1; WP_042450108.1; WP_007735391.1; WP_065352885.1; WP_007727244.1; WP_065352214.1; and WP_003943732.1.

In some embodiments, the hydrocarbon-utilizing microorganism is characterized by having increased metabolic pathway activity, wherein the pathway is any one of pathways 1 to 7 of Table 2, or any combination thereof. In some embodiments, a hydrocarbon-utilizing microorganism is characterized by having overexpression of at least one gene selected from: WP_003945297.1; WP_065352723.1; WP_065352722.1; WP_065352721.1; WP_050656813.1; WP_007726140.1; WP_007726141.1; WP_065351823.1; WP_003941324.1; WP_019749215.1; WP_003941638.1; WP_042450649.1; WP_003941322.1; WP_065351660.1; WP_003945295.1; WP_042450854.1; WP_065351492.1; WP_003940783.1; WP_065351661.1; WP_065352563.1; WP_065351754.1; WP_030536487.1; WP_065351402.1; WP_080726744.1; WP_042449699.1; WP_065351224.1; WP_003941275.1; WP_003939932.1; WP_007729732.1; WP_003946304.1; WP_042452232.1; WP_007726566.1; WP_054187331.1; WP_003941308.1; WP_007726568.1; WP_058227688.1; WP_065351782.1; WP_065352072.1; WP_020968150.1; WP_003941552.1; WP_065352440.1; WP_003942314.1; WP_042450108.1; WP_007735391.1; WP_065352885.1; WP_007727244.1; WP_065352214.1; and WP_003943732.1, wherein the hydrocarbon-utilizing microorganism is further characterized by having any one of: (i) increased metabolic pathway activity, wherein the pathway is any one of pathways 1 to 7 of Table 2, and any combination thereof, (ii) nonspecific dehydrogenation activity, (iii) low fatty acid oxidation activity or being devoid of fatty acid oxidation activity, or a combination thereof.

In some embodiments, a hydrocarbon-utilizing microorganism of the invention nonspecifically dehydrogenases an alkane as a main pathway for energy harvest.

In some embodiments, a hydrocarbon-utilizing microorganism of the invention is characterized by a low fatty acid oxidation activity.

In some embodiments, a hydrocarbon-utilizing microorganism of the invention is devoid of fatty acid oxidation activity.

As used herein, the phrase “low oxidation activity” refers to that the hydrocarbon-utilizing microorganism preferentially performs dehydrogenation of an alkane over fatty acid oxidation, as the main pathway for energy source.

In some embodiments, “preferentially” refers to the ratio of alkane dehydrogenation rate over fatty acid oxidation rate, the number or mole of alkane molecules being dehydrogenated over the number or mole of fatty acid molecules being oxidized.

As used herein, “preferentially” comprises at least 2-fold, at least 3-fold, at least 4-fold, at least 5-fold, at least 7-fold, at least 9-fold, or at least 10-fold more, or any value and range therebetween. Each possibility represents a separate embodiment of the invention. In some embodiments, preferentially comprises 2-6-fold, 2-8-fold, 2-10-fold, 3-8-fold, 4-12-fold, 10-50-fold, 22-65-fold, or 8-35-fold more. Each possibility represents a separate embodiment of the invention.

In some embodiments, the method comprises determining a microorganism is suitable for use as hydrocarbon-utilizing microorganism, comprising a step of determining the microorganism has any one of: (i) overexpression of at least one gene selected from: WP_003945297.1; WP_065352723.1; WP_065352722.1; WP_065352721.1; WP_050656813.1; WP_007726140.1; WP_007726141.1; WP_065351823.1; WP_003941324.1; WP_019749215.1; WP_003941638.1; WP_042450649.1; WP_003941322.1; WP_065351660.1; WP_003945295.1; WP_042450854.1; WP_065351492.1; WP_003940783.1; WP_065351661.1; WP_065352563.1; WP_065351754.1; WP_030536487.1; WP_065351402.1; WP_080726744.1; WP_042449699.1; WP_065351224.1; WP_003941275.1; WP_003939932.1; WP_007729732.1; WP_003946304.1; WP_042452232.1; WP_007726566.1; WP_054187331.1; WP_003941308.1; WP_007726568.1; WP_058227688.1; WP_065351782.1; WP_065352072.1; WP_020968150.1; WP_003941552.1; WP_065352440.1; WP_003942314.1; WP_042450108.1; WP_007735391.1; WP_065352885.1; WP_007727244.1; WP_065352214.1; and WP_003943732.1, (ii) increased metabolic pathway activity, wherein the pathway is any one of pathways 1 to 7 of Table 2, and any combination thereof, (iii) nonspecific dehydrogenation activity, (iv) low fatty acid oxidation activity or being devoid of fatty acid oxidation activity, or a combination thereof.

In some embodiments, the hydrocarbon-utilizing microorganism is characterized by having overexpression of a plurality of genes involved in fatty acid metabolism compared to control.

As used herein, a plurality comprises 2 or more, 4 or more, 6 or more, or 10 or more, or any value and range therebetween. Each possibility represents a separate embodiment of the invention. In some embodiments, a plurality comprises 2-7, 3-14, 5-20, or 2-30. Each possibility represents a separate embodiment of the invention.

In some the hydrocarbon-utilizing microorganism is an isolated microorganism or a synthetically produced microorganism. In some the hydrocarbon-utilizing microorganism is a recombinant microorganism. In some embodiments, the hydrocarbon-utilizing microorganism is a mutated or a mutant microorganism.

In some embodiments, a control is a wild type microorganism. In some embodiments, the control is a microorganism characterized by having at least 10-fold less expression of at least one gene selected from: WP_003945297.1; WP_065352723.1; WP_065352722.1; WP_065352721.1; WP_050656813.1; WP_007726140.1; WP_007726141.1; WP_065351823.1; WP_003941324.1; WP_019749215.1; WP_003941638.1; WP_042450649.1; WP_003941322.1; WP_065351660.1; WP_003945295.1; WP_042450854.1; WP_065351492.1; WP_003940783.1; WP_065351661.1; WP_065352563.1; WP_065351754.1; WP_030536487.1; WP_065351402.1; WP_080726744.1; WP_042449699.1; WP_065351224.1; WP_003941275.1; WP_003939932.1; WP_007729732.1; WP_003946304.1; WP_042452232.1; WP_007726566.1; WP_054187331.1; WP_003941308.1; WP_007726568.1; WP_058227688.1; WP_065351782.1; WP_065352072.1; WP_020968150.1; WP_003941552.1; WP_065352440.1; WP_003942314.1; WP_042450108.1; WP_007735391.1; WP_065352885.1; WP_007727244.1; WP_065352214.1; and WP_003943732.1.

As used herein, the term “overexpression” encompasses an increased expression of at least 5%, at least 15%, at least 35%, at least 45%, at least 65%, at least 80%, at least 100%, at least 200%, at least 350%, at least 500%, at least 750%, at least 850%, or at least 1,000%, or any value and range therebetween. Each possibility represents a separate embodiment of the invention. In some embodiments, overexpression comprises an increased expression of 5-200%, 75-300%, 5-60%, 1-50%, 150-650%, 275-750%, 500-860%, 700-950%, or 800-1,000%.

Methods for determining and quantifying the level of gene overexpression are common and would be apparent to one of ordinary skill in the art. Non-limiting examples include, but are not limited to, PCR, quantitative PCR, e.g., real-time PCR, next generation sequencing, and others.

In some embodiments, the hydrocarbon-utilizing microorganism is Rhodococcus mutant strain KSM-B-3M.

As used herein, the term “microorganism” means prokaryotic and eukaryotic microbial species from the domains Archaea, Bacteria and Eucarya, the latter including yeast and filamentous fungi, protozoa, algae, or higher Protista.

### The Method

According to an aspect of some embodiments of the present invention there is provided a method for selectively functionalizing alkanes, which comprises a step of (a) providing an alkane; (b) dehydrogenating the alkane by a hydrocarbon-utilizing microorganism; followed by (c) isomerization-hydrofunctionalization reaction; thereby yielding a functionalized alkane.

In some embodiments, the step (b) is performed in an aqueous sterile medium consisting of phosphate buffer, an amino acid, thiamine hydrochloride and magnesium sulfate heptahydrate or any combination thereof. In some embodiments, the step (b) is performed in an aqueous sterile medium consisting of distilled water, KH₂PO₄, K₂HPO₄, monosodium glutamate monohydrate, thiamine hydrochloride and magnesium sulfate heptahydrate.

In some embodiments, the yield of step (b) dehydrogenating the alkane by a hydrocarbon-utilizing microorganism is more than 18%, is more than 19%, is more than 20%, is more than 25%, is more than 30%, is more than 35%, is more than 40%, is more than 45%, is more than 50%, is more than 55% is more than 60%, is more than 65%, is more than 70%, is more than 75%, is more than 80%, or is more than 85%. In some embodiments, the yield may be determined to the corrected yield calculated from the recovered mass obtained after purification multiplied by the ratio of the surface area of the corresponding peaks alkene/(alkane+alkene) measured by GC.

In some embodiments, the step (b) leads to the formation of at least two alkenes. In some embodiments, at least 2 alkenes is 3 alkenes.

In some embodiments, the dehydrogenation ratio between at least two alkenes is more than 25%, more than 30%, more than 31%, more than 32%, more than 35%, more than 40%, more than 45%, more than 48%, more than 50%, more than 52%, more than 55%, more than 57%, more than 60%, more than 63%, more than 65%, more than 68%, more than 70%, more than 72%, more than 74%, more than 77%, more than 79%, more than 80%, more than 83%, more than 85%, more than 89%, more than 90%, more than 93%, more than 95%, or more than 98%.

In some embodiments, step (b) is performed at a temperature ranging from 20° C. to 34° C. In some embodiments, step (b) is performed at a temperature ranging from 26° C. to 34° C. In some embodiments, step (b) is performed at a temperature ranging from 28° C. to 30° C. In some embodiments, step (b) is performed at a temperature ranging from 28° C. to 34° C.

In some embodiments, step (b) is performed at a temperature of 20° C., 21° C., 22° C., 23° C., 24° C., 25° C., 26° C., 27° C., 28° C., 29° C., 30° C., 31° C., 32° C., 33° C., or 34° C., including any value and range therebetween.

In some embodiments, the pH of the aqueous medium is within the range of 6.0 to 6.8. In some embodiments, the pH of the aqueous medium is within the range of 6.3 to 6.8. In some embodiments, the pH of the aqueous medium is within the range of 6.3 to 6.5.

In some embodiments, the pH of the aqueous medium is about 6.0, about 6.1, about 6.2, about 6.3, about 6.4, about 6.5, about 6.6, about 6.7, or about 6.8, including any value and range therebetween.

In some embodiments, step (b) is performed for at least 5 days. In some embodiments step (b) is performed for 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, or 45 days, including any value and range therebetween.

In some embodiments, step (c) is metal assisted isomerization-hydrofunctionalization reaction.

In some embodiments, the isomerization-hydrofunctionalization comprises a reaction selected from, without limitation halogenolysis, oxidation, copper-catalyzed allylation, hydroboration, hydrosilylation, hydrozirconation, hydroarylation hydroamination, olefin cross-metathesis, cycloadditions, dimetalation and allylic C—H functionalization.

In some embodiments, step (c) affords the formation of a new terminal covalent bond such as C—C, C—O, C—X wherein X is halogen, C—Si, C—N, C—B, C—P, C—Se, C—Zn, or any combination thereof.

In some embodiments, step (a) comprises dehydrogenation reaction mediated by hydrocarbon-utilizing microorganism; followed by recovering the microorganism; and re-dissolving the microorganism in aqueous medium and perform another dehydrogenation reaction.

In some embodiments, the microorganism was recovered after 5 days, after 6 days, after 7 days, after 8 days, after 9 days, after 10 days, after 11 days, after 12 days, after 13 days, after 14 days, after 15 days, after 16 days, after 17 days, after 18 days, after 19 days, after 20 days, after 21 days, after 22 days, after 23 days, after 24 days, after 25 days, 26 days, 27 days, or 28 days, including any value and range therebetween.

In some embodiments, the microorganism was recovered after at least 5 days, at least 10 days, at least 15 days, at least 20 days, at least 25 days, or at least 28 days, including any value and range therebetween.

According to another embodiment, the present invention provides a composition comprising an alkane and at least two cis-alkene regioisomers, wherein the ratio of the at least two cis-alkenes to the alkane is in a range of 95:5 to 80:20. In some embodiments, the ratio of the at least two cis-alkenes to the alkane is in a range of 95:5 to 90:10. In some embodiments, the ratio of the at least two cis-alkenes to the alkane is in a range of 95:5 to 93:7. In some embodiments, the ratio of the at least two cis-alkenes to the alkane is in a range of 90:10 to 80:20.

In some embodiments the ratio of the at least two cis-alkenes to the alkane is about 95:5. In some embodiments the ratio of the at least two cis-alkenes to the alkane is about 93:7. In some embodiments the ratio of the at least two cis-alkenes to the alkane is about 90:10. In some embodiments the alkane the at least two cis-alkene regioisomers in a ratio of about 85:15. In some embodiments the ratio of the at least two cis-alkenes to the alkane is about 83:17. In some embodiments the ratio of the at least two cis-alkenes to the alkane is about 80:20.

### Definitions

As used herein, the term “alkyl” describes an aliphatic hydrocarbon including straight chain and branched chain groups. In some embodiments, the alkyl group has 4 to 100 carbon atoms, and in some embodiments, 4-40 carbon atoms. Whenever a numerical range; e.g., “4-100”, is stated herein, it implies that the group, in this case the alkyl group, may contain 4 carbon atom, 5 carbon atoms, 6 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule. Additional substituents are also contemplated

The term “halide”, “halogen” or “halo” describes fluorine, chlorine, bromine or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “sulfinyl” group describes an —S(═O)—R′ group, where R′ is as defined herein.

A “sulfonyl” or “sulfonate” group describes an —S(═O)₂—R′ group, where R′ is as defined herein.

A “carbamyl” or “carbamate” group describes an —OC(═O)—NR′R″ group, where R′ is as defined herein and R″ is as defined for R′.

A “nitro” group refers to a —NO₂ group.

A “cyano” or “nitrile” group refers to a group.

As used herein, the term “azido” group refers to —N₃.

The term “sulfonamide” refers to a —S(═O)₂—NR′R″ group, with R′ and R″ as defined herein.

The term “phosphonyl” or “phosphonate” describes an —O—P(═O)(OR′)₂ group, with R′ as defined hereinabove.

The term “phosphinyl” describes a —PR′R″ group, with R′ and R″ as defined hereinabove.

The term “alkaryl” describes an alkyl, as defined herein, which substituted by an aryl, as described herein. An exemplary alkaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

### General:

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

Unless stated otherwise, all tools used in contact with bacterial strains were sterile or sterilized prior use and all manipulation in their presence were conducted next to a gas flame. The mutant strain KSM-B-3M was obtained from the Japanese National Institute of Technology and Evaluation (NITE) under the code name FERM BP-1531 and with the consent of the main depositor of the original patent (Dr. Kenzo Koike, U.S. Pat. No. 5,059,532). Throughout this study, precautions of biosafety level 1 were taken regarding this specific strain and, to our knowledge, no harm to the people working directly on the project was observed during the course of our study.

Unless stated otherwise, chemically based reactions were conducted in flame-dried glassware under a positive pressure of argon. Ether and THF were dried from Pure-Sols® Purification System (Innovative Technology©). All other commercially obtained reagents were used as received. Thin-layer chromatography (TLC) was conducted with E. Merck silica gel 60 F254 pre-coated plates, (0.25 mm) and visualized by exposure to UV light (254 nm) or stained with anisaldehyde, phosphomolybdic acid or potassium permanganate. Column chromatography was performed using Fluka silica gel 60 Å (40-63 mm, 230-400 mesh). NMR spectra were recorded on Bruker spectrometers (AVIII400) and are reported relative to residual deuterated solvent signals. Chemical shifts are reported in parts per million (ppm) with respect to the residual solvent signal CDCl₃ NMR: δ=7.26; ¹³C NMR: 6=77.16). Peak multiplicities are reported as follows: s=singlet, bs=broad singlet, d=doublet, t=triplet, dd=doublet of doublets, td=triplet of doublets, m=multiplet. High-resolution mass spectra (HRMS) were obtained by the mass spectrometry facility of the Technion. Reactions were monitored by gas chromatography spectrometry (GC) using an Agilent Technologies 7820A GC with an Agilent Technologies 19091J-413 (30 m×0.3 mm) column.

Growth of Rhodococcus KSM-B-3M cells and setting alkane dehydrogenation systems: Rhodococcus sp. strain KSM-B-3M cells were grown in a nutrient broth solution (NB n° 3; Sigma Aldrich, Israel cat. 70149) for 20-24 h at 30° C. and under agitation of 180 rpm. 1 mL of bacterial culture was sub-cultured in fresh NB (200 mL) for an additional 20-24 h incubation at 30° C. and under agitation of 180 rpm. Bacterial cells were sedimented at 4800 g for 10 min, and washed with saline to remove medium leftovers. 1 gram of bacterial pellet was transferred to a 250 Erlenmeyer vessel and suspended in 57.5 ml medium A (0.464 M KH₂PO₄, 0.324M K₂HPO₄, 21.3 mM monosodium glutamate monohydrate, 0.3 mM thiamine hydrochloride and 0.4 mM magnesium sulfate heptahydrate in dH₂O) supplemented with 8.5 mmol of alkane. Erlenmeyers were incubated at 31.5° C. under agitation of 180 rpm.

DNA extraction and phylogenetic analyses of Rhodococcus strains: Gram-positive bacteria treatment of DNeasy Blood and Tissue kit (QIAGEN, Hilden, Germany) was used as the first step of the DNA extraction protocol, with slight modifications prior to cell lysis. Briefly, 100 mL of NB overnight KSM-B-3M cultures were centrifuged at 4,800×g for 5 min at 15° C., and the pellet was stored at −20° C. for 1 h. The thawed pellet was suspended with 200 μL cell lysis containing: 20 mM Tris-Cl at pH=8, 2 mM sodium EDTA, 1.2% Trition X-100, and 20 mg lysozyme, and incubated at 37° C. for 1 h. Then, samples were treated using the DNeasy protocol, according to the manufacturer's instructions.

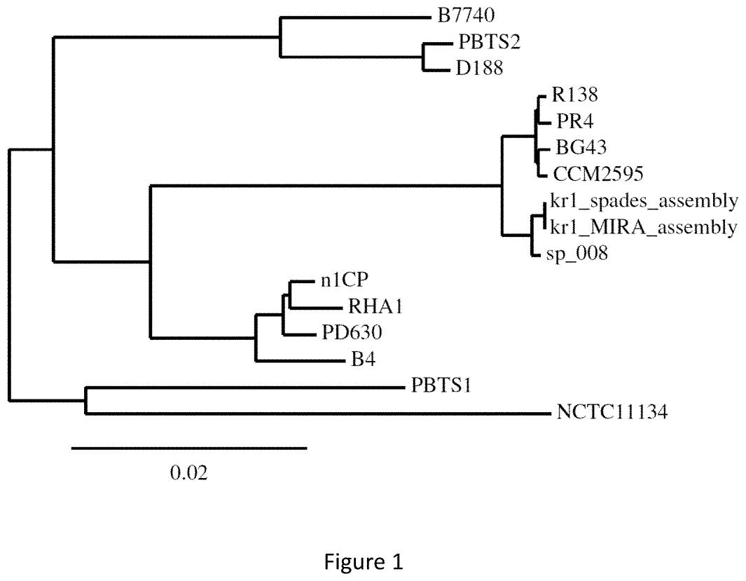

To identify the closest Rhodococcus strain to KSM-B-3M (assigned to as krl) whole genome sequencing was performed and two assemblies of KSM-B-3M were generated using SPAdes (Nurk et.al. 2013) and Mira (Chevreux et.al 2004). We used RealPhy (Bertels et.al. 2014) to reconstruct a phylogenetic tree that includes two assemblies of KSM-B-3M (krl_spades assembly, krl_MIRA assembly) and 14 publicly available genomes. Mapping the reads was performed with bowtie2 (Langmead, B. and Salzberg, S. L. 2012). PhyML was used to build the tree (Guindon et. al. 2010). Dendroscope (D. H. Huson and C. Scornavacca 2010) was used to generate a phylogram visualization of the tree (FIG. 1).

### Example 1

### Optimization of the Rhodococcus Biocatalytic Dehydrogenation of Hexadecane

Table 1 shows the parameters were optimized to lead to the general procedure for the dehydrogenation reaction. The time of comparison of all gas chromatography (GC) conversions was arbitrarily determined after 7 days.

MSG=Monosodium glutamate monohydrate, nd=not determined

| | Quantity | | | | | |
|---|---|---|---|---|---|---|
| | wet cells | Volume | Total | | | Phosphate |
| | KSM-3- | hexadecane | volume | Erlenmeyer | Temperature | buffer |
| Entries | BM (g) | (mL) | (mL) | (mL) | (° C.) | (mol/L) |
| 1 | 1 | 5 | 25 | 250 | 26 | 0.25 |
| 2 | 1 | 5 | 25 | 250 | 30 | 0.25 |
| 3 | 1 | 10 | 25 | 250 | 30 | 0.25 |
| 4 | 1 | 2.5 | 25 | 250 | 30 | 0.25 |
| 5 | 1 | 2.5 | 25 | 250 (rigged | 30 | 0.25 |
| | | | | flask) | | |
| 6 | 1 | 1.25 | 25 | 250 | 30 | 0.25 |
| 7 | 2 | 5 | 25 | 250 | 30 | 0.25 |
| 8 | 1 | 2.5 | 25 | 250 | 28 | 0.25 |
| 9 | 1 | 2.5 | 25 | 250 | 32 | 0.25 |
| 10 | 1 | 2.5 | 25 | 250 | 34.5 | 0.25 |
| 11 | 1 | 2.5 | 60 | 250 | 30 | 1 |
| 12 | 1 | 2.5 | 60 | 250 | 30 | 2 |
| 13 | 1 | 2.5 | 100 | 500 | 30 | 1 |
| 14 | 1 | 2.5 | 60 | 250 | 30 | 0.25 |
| 15 | 1 | 2.5 | 60 | 250 | 30 | 1 |
| 16 | 1 | 2.5 | 60 | 250 | 30 | 1 |
| 17 | 1 | 2.5 | 60 | 250 | 30 | 1 |
| 18 | 1 | 2.5 | 60 | 250 | 30 | 0.8 |
| 19 | 1 | 2.5 | 60 | 250 | 30 | 1 |
| 20 | 1 | 2.5 | 60 | 250 | 30 | 1 |
| 21 | 1 | 2.5 | 60 | 250 | 30 | 1 |

| | | | | | GC conversion |
|---|---|---|---|---|---|
| | Amino acid | Thiamine•HCl | MgSO•7H₂O | | alkene/alkane |
| Entries | (g/L) | (g/L) | (g/L) | pH | (%, 7 days) |
| 1 | MSG | 0.1 | 0.1 | nd | 5.9 |
| | (1 g/L) | | | | |
| 2 | MSG | 0.1 | 0.1 | nd | 11.7 |
| | (1 g/L) | | | | |
| 3 | MSG | 0.1 | 0.1 | nd | 3.8 |
| | (1 g/L) | | | | |
| 4 | MSG | 0.1 | 0.1 | nd | 33.4 |
| | (1 g/L) | | | | |
| 5 | MSG | 0.1 | 0.1 | nd | 5.3 |
| | (1 g/L) | | | | |
| 6 | MSG | 0.1 | 0.1 | nd | 13.5 |
| | (1 g/L) | | | | |
| 7 | MSG | 0.1 | 0.1 | nd | 21.7 |
| | (1 g/L) | | | | |
| 8 | MSG | 0.1 | 0.1 | nd | 11.8 |
| | (1 g/L) | | | | |
| 9 | MSG | 0.1 | 0.1 | nd | 5.5 |
| | (1 g/L) | | | | |
| 10 | MSG | 0.1 | 0.1 | nd | 0 |
| | (1 g/L) | | | | |
| 11 | MSG | 0.4 | 0.4 | nd | 43.0 |
| | (4 g/L) | | | | |
| 12 | MSG | 0.8 | 0.8 | nd | 0 |
| | (8 g/L) | | | | |
| 13 | MSG | 0.4 | 0.4 | nd | 19.0 |
| | (4 g/L) | | | | |
| 14 | MSG | 0.4 | 0.4 | nd | 37.0 |
| | (4 g/L) | | | | |
| 15 | MSG | 0.4 | 0.4 | nd | 10.9 |
| | (1 g/L) | | | | |
| 16 | MSG | 0.1 | 0.4 | nd | 35.8 |
| | (4 g/L) | | | | |
| 17 | MSG | 0.4 | 0.1 | nd | 34.3 |
| | (4 g/L) | | | | |
| 18 | MSG | 0.1 | 0.1 | 6.4 | 49.1 |
| | (4 g/L) | | | | |
| 19 | Glycine | 0.4 | 0.1 | nd | 0.7 |
| | (4 g/L) | | | | |
| 20 | Glycine | 0.4 | 0.1 | 5.8 | 0 |
| | (4 g/L) | | | | |
| 21 | Glycine | 0.4 | 0.1 | 7 | 0 |
| | (4 g/L) | | | | |

### Example 2

### General Procedure for the Dehydrogenation Reaction Mediated by Rhodococcus Mutant Strain KSM-B-3M

Prior to the dehydrogenation reaction, an aqueous medium was freshly prepared by typically dissolving into 1 L of distilled water KH₂PO₄ (67.3 g, 0.495 M), K₂HPO₄ (53.1 g, 0.305 M), monosodium glutamate monohydrate (4 g, 21.3 mM), thiamine hydrochloride (0.1 g, 0.3 mM) and magnesium sulfate heptahydrate (0.1 g, 0.4 mM) and stirring the solution for 10 min at room temperature. After complete dissolution of all the salts, the solution (pH=6.4) was sterilized.

In a sterile 250 mL Erlenmeyer (large entry) bearing a breathable cork, 1 g of wet cells KSM-B-3M was dissolved into 57.5 mL of this sterile medium. After addition of the alkane (8.5 mmol), the resulting solution was stirred inside a shaker (180 rpm, 30° C.) over the course of several days under aerobic conditions. During that time, the evolution of the transformation (progressively turning to an orange slurry suspension) was monitored by GC analyses of 0.5 mL aliquots containing both phases. After reaction, the phases were separated, and the aqueous phase was extracted with EtOAc (3×20 mL). The combined organic phases were dried over MgSO₄ anhydrous and concentrated under reduce pressure. The desired products were purified through a short pad of silica gel (100% hexane to 1% Et₂O/hexane) for analyses.

### Example 3

### Scope of the Dehydrogenation Reaction Mediated by Rhodococcus Mutant Strain KSM-B-3M

Using the general procedure of Example 2 for the dehydrogenation reaction mediated by Rhodococcus mutant strain KSM-3-BM, several alkanes and other alkyl derivatives (8.5 mmol of substrate) were tested. Exception: eicosane (8.5 mmol) was dissolved into dodecane (8.5 mmol). Following the general procedure and the purification step and after reaction, dodecane was distilled under vacuum to yield the desired mixture of eicosenes and remaining eicosane.

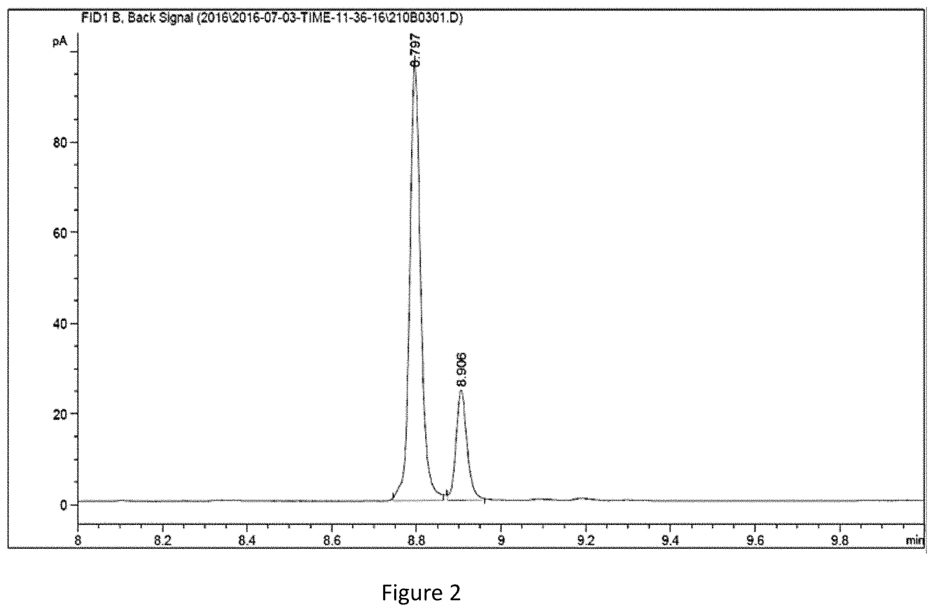

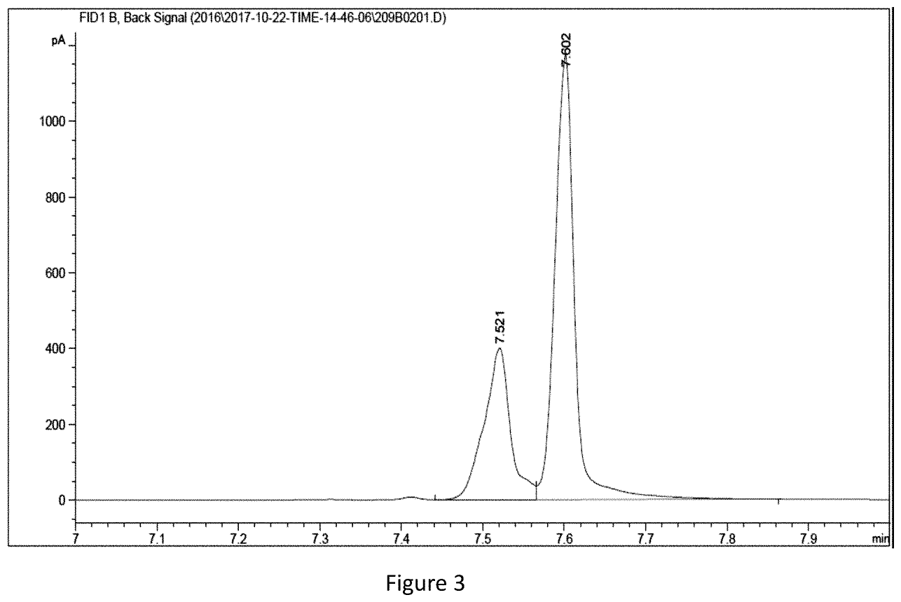

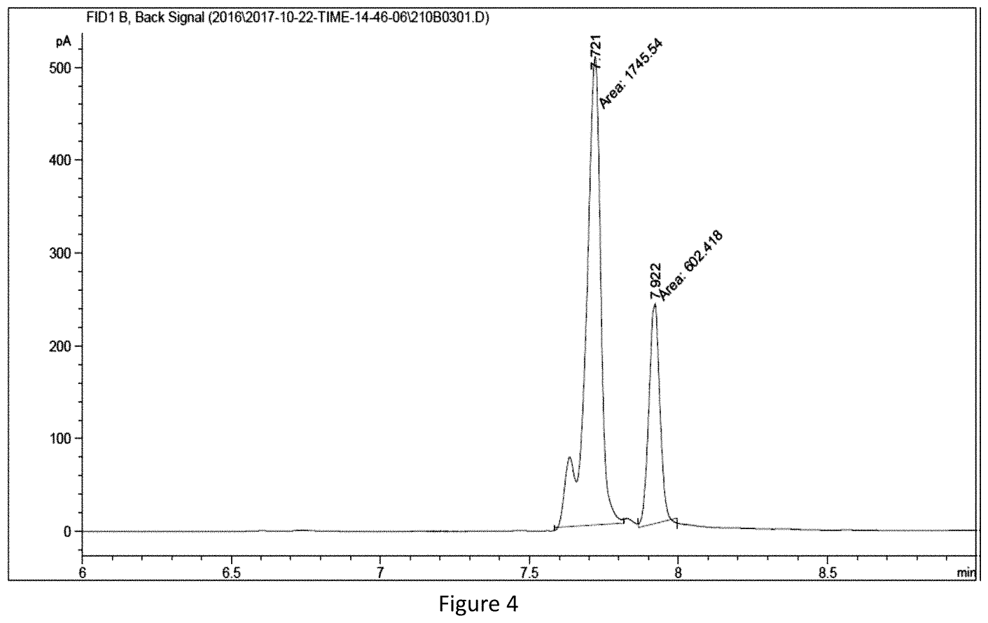

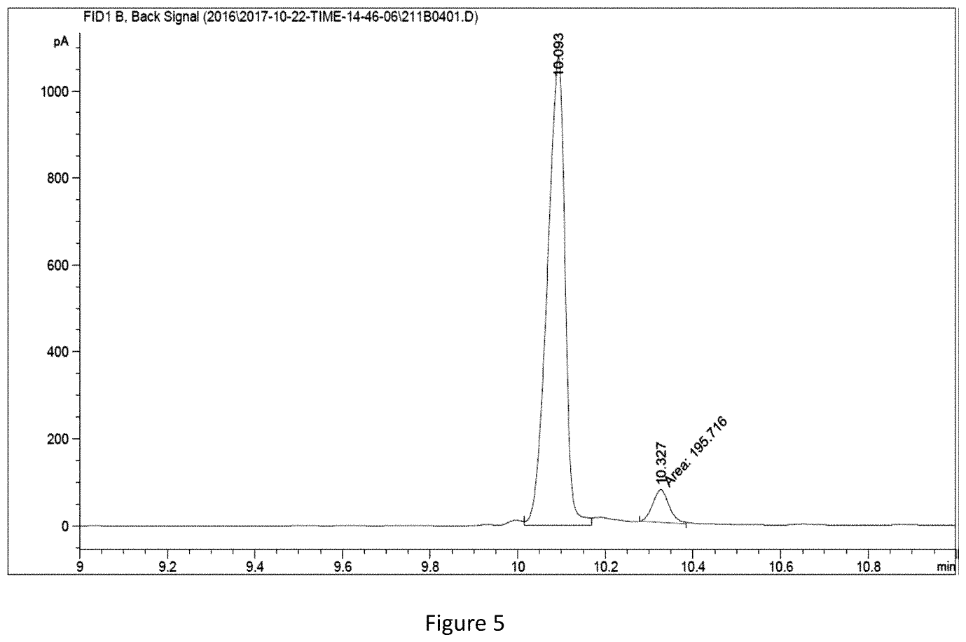

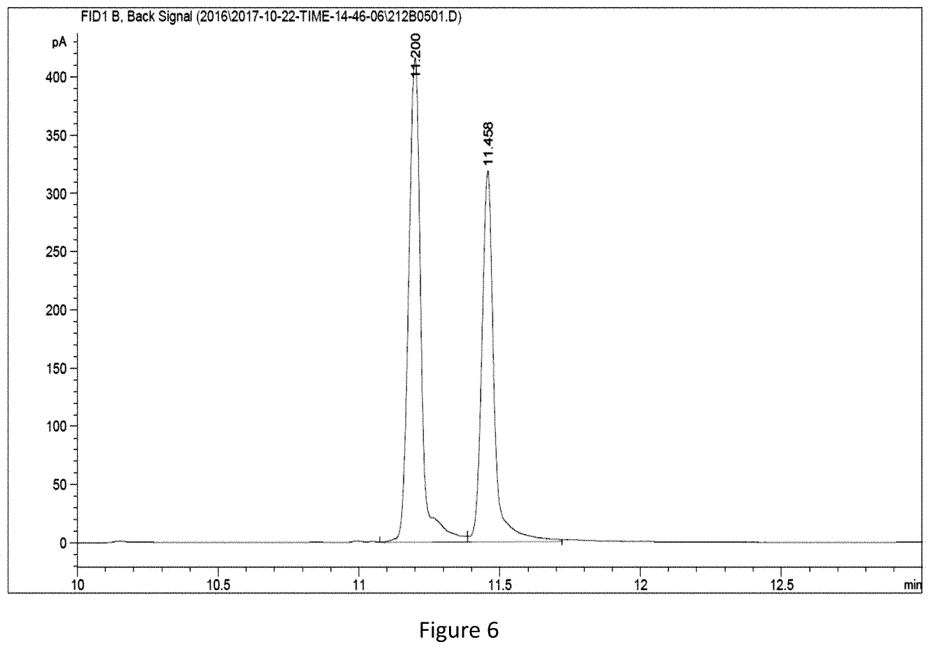

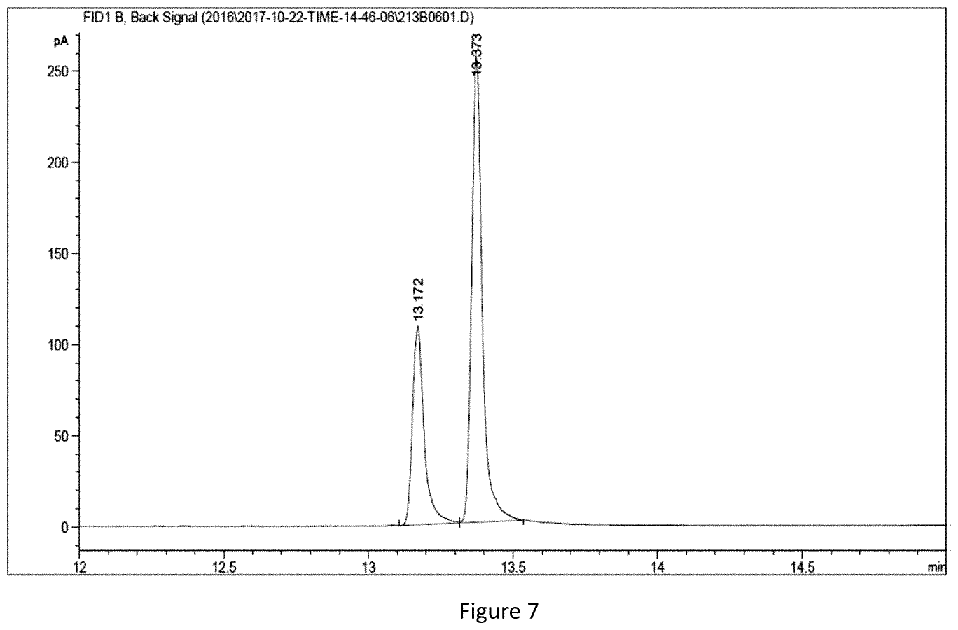

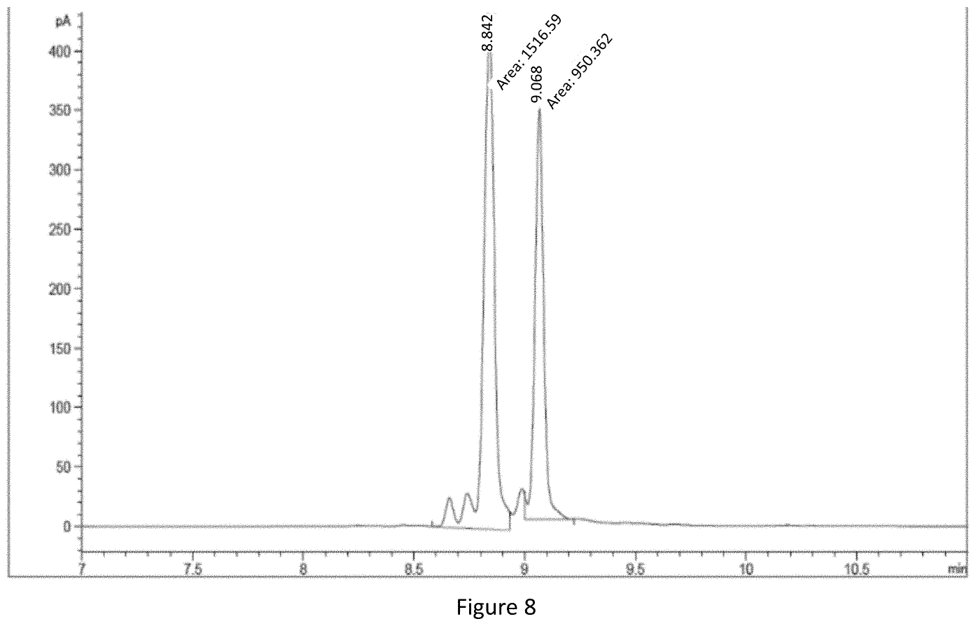

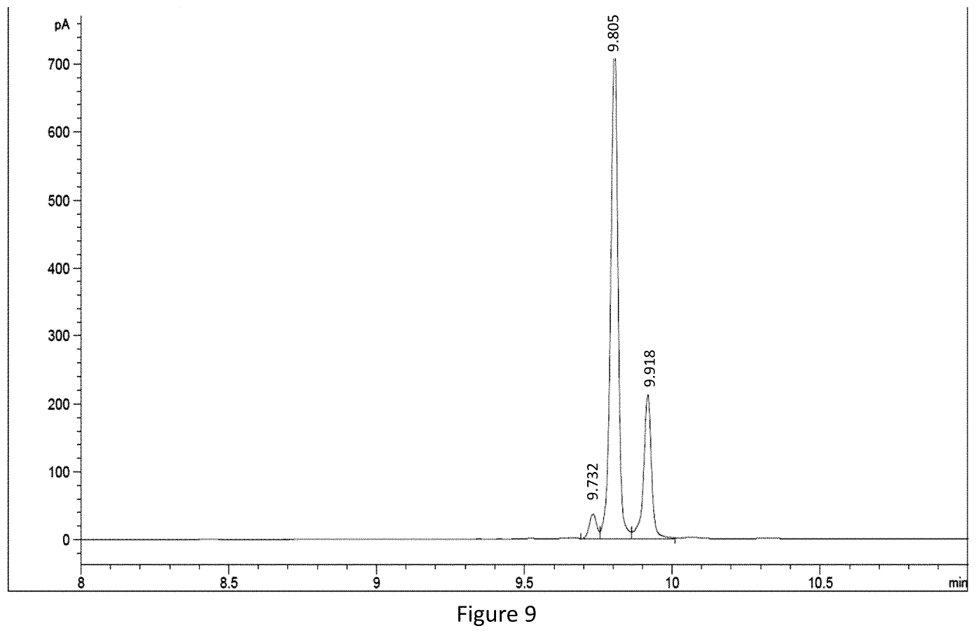

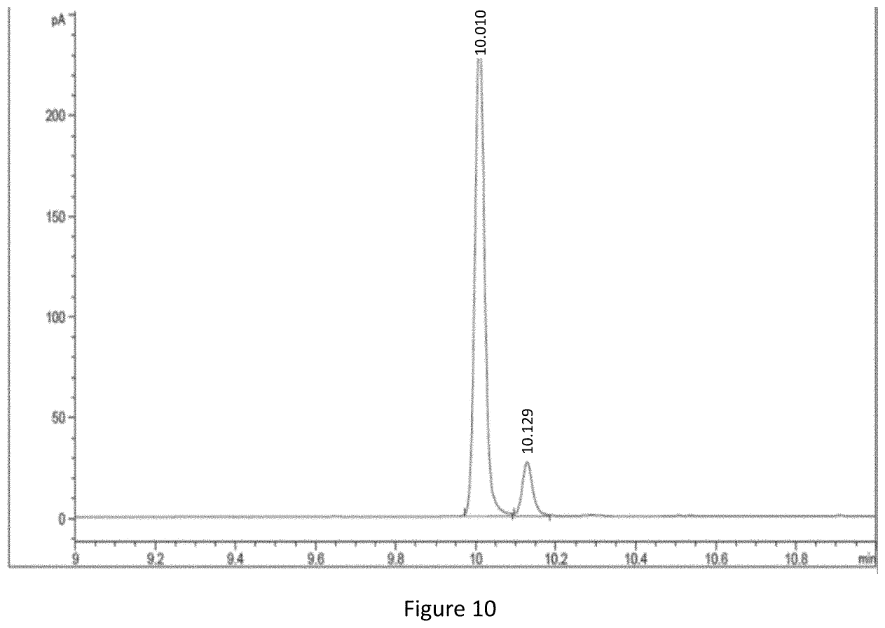

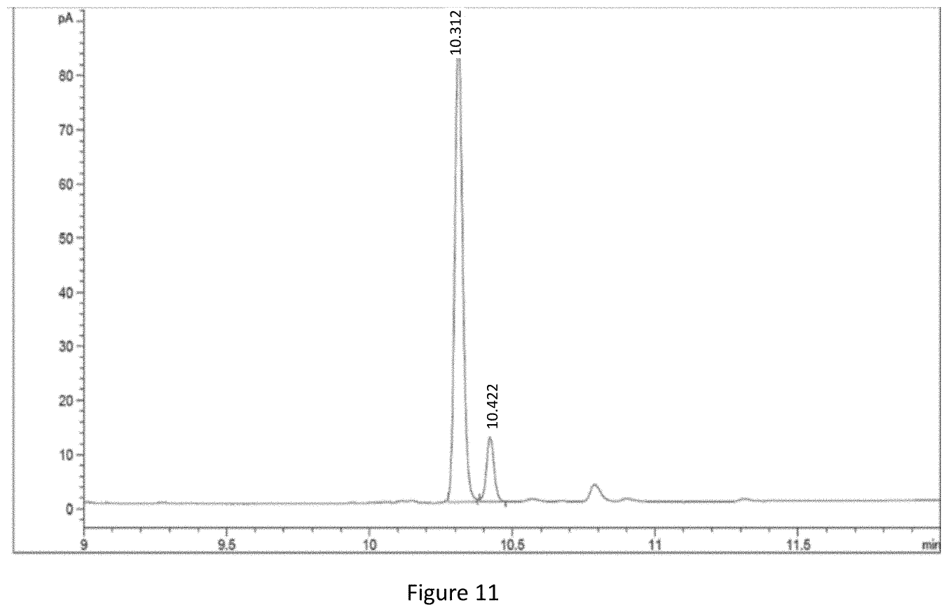

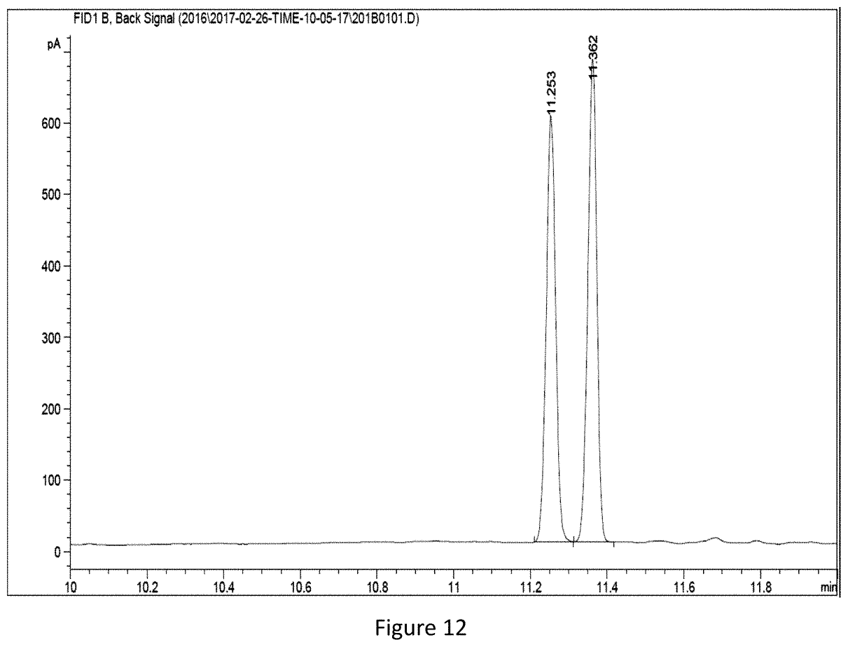

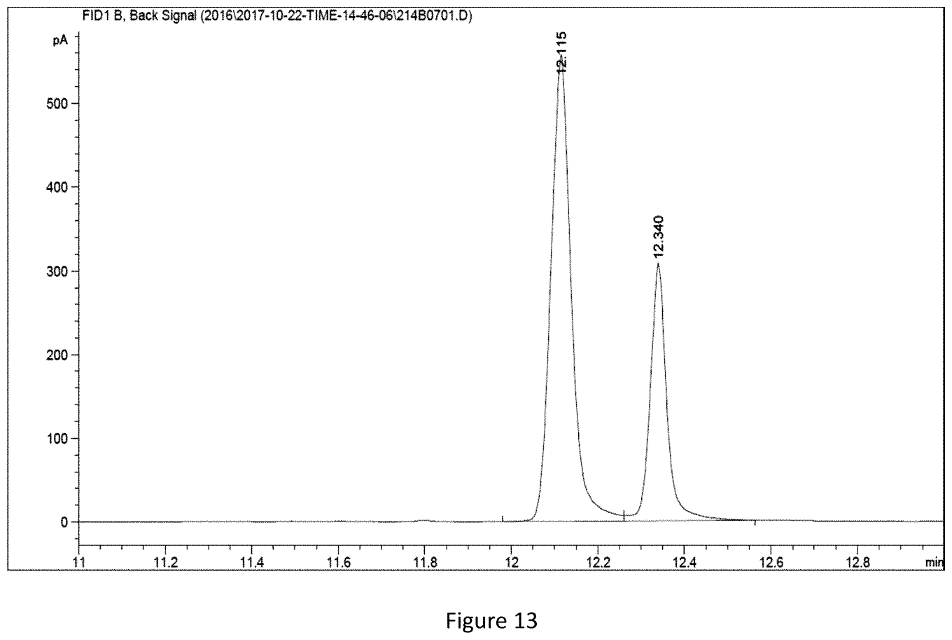

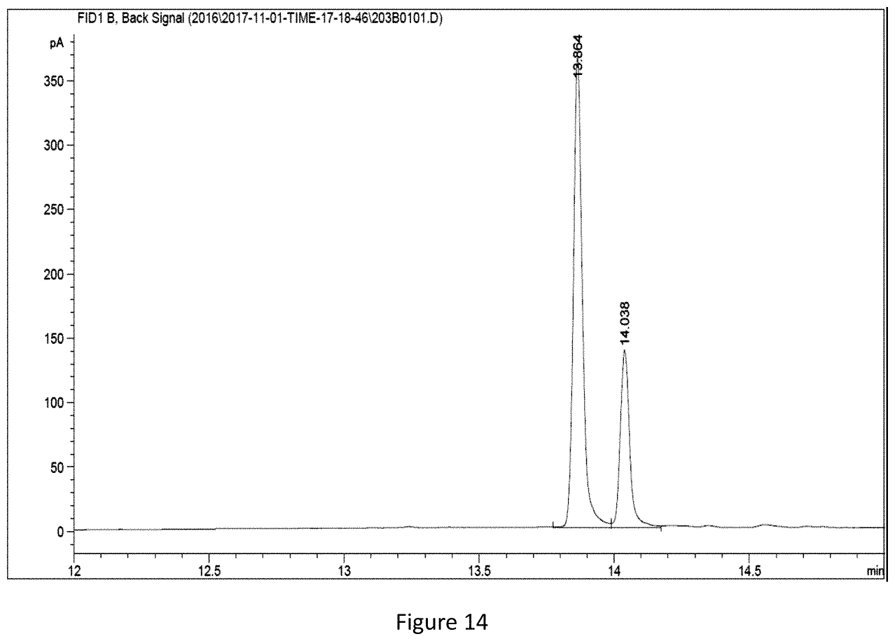

All alkenes could not be separated from their saturated precursors and were described as mixtures. The dehydrogenation ratio refers to the ratio alkenes/(alkane+alkenes), and the ratios were evaluated by GC (FIGS. 2-14). Reported yields were corrected according to these dehydrogenation ratios. In all reported cases, only the Z resulting olefins were observed by NMR (Z/E>99:1).

High productivity (GC yield >15% after 10 days)

cis-hexadecenes (1): On a 25.5 mmol scale, corrected yield=3453 mg (61%). Dehydrogenation ratio (GC)=83%. Colorless liquid. Z/E>99:1. Mixture: ¹H NMR (400 MHz, CDCl₃) δ 5.37 (t, J=4.6 Hz, 2H), 2.05 (dd, J=11.9, 6.2 Hz, 4H), 1.29 (m, 21H), 0.91 (t, J=6.6 Hz, 3H). ¹³C NMR (101 MHz, CDCl₃) δ 130.0, 32.2, 32.1, 30.0, 30.0, 30.0, 29.8, 29.6, 29.6, 29.3, 27.4, 22.9, 22.9, 14.3. HRMS (APCI-MS ES+) [M−H]+, calculated for C₁₆H₃₃; 249.2556; found 249.2582.

cis-tetradecenes (2): Corrected yield=283 mg (17%). Dehydrogenation ratio (GC)=31%. Colorless liquid. Z/E >99:1. Mixture: ¹H NMR (400 MHz, CDCl₃) δ 5.37 (t, J=4.7 Hz, 2H), 2.16-1.97 (m, 4H), 1.46-1.16 (m, 16H), 0.91 (t, J=6.6 Hz, 6H). 13C NMR (101 MHz, CDCl₃) δ 130.1, 130.1, 130.0, 32.3, 32.2, 32.2, 30.1, 30.0, 30.0, 29.8, 29.7, 29.6, 29.6, 27.5, 27.2, 23.0, 22.6, 14.3, 14.2. HRMS (APCI-MS ES+) [M−H]+, calculated for C₁₄H₂₉; 197.2269; found 197.2278.

cis-pentadecenes (3): Corrected yield=860 mg (48%). Dehydrogenation ratio (GC)=74%. Colorless liquid. Z/E>99:1. Mixture: ¹H NMR (400 MHz, CDCl₃) δ 5.37 (t, J=4.6 Hz, 2H), 2.11-2.01 (m, 4H), 1.41-1.24 (m, 18H), 0.96-0.87 (m, 6H). ¹³C NMR (101 MHz, CDCl₃) δ 130.3, 130.0, 128.2, 32.2, 32.3, 32.1, 32.1, 31.8, 30.0, 30.0, 29.9, 29.8, 29.7, 29.6, 29.6, 29.6, 29.6, 29.5, 29.5, 29.2, 27.4, 27.4, 22.9, 22.9, 22.9, 22.8, 14.2, 14.2. HRMS (APCI-MS ES+) [M−H]+, calculated for C₁₅H₃₁; 211.2426; found 211.2401.

cis-heptadecenes (4): Corrected yield=1180 mg (58%). Dehydrogenation ratio GC=93%. Colorless liquid. Z/E>99:1. Mixture: ¹H NMR (400 MHz, CDCl₃) δ 5.43-5.34 (m, 2H), 2.11-2.00 (m, 4H), 1.46-1.22 (m, 22H), 0.93 (t, J=6.8 Hz, 6H). ¹³C NMR (101 MHz, CDCl₃) δ 130.0, 32.2, 32.2, 30.1, 29.9, 29.6, 29.6, 29.6, 29.6, 27.5, 23.0, 14.3. HRMS (APCI-MS ES+) [M−H]+, calculated for C₁₇H₃₅; 239.2739; found 239.2754.

cis-octadecenes (5): Corrected yield=735 mg (34%). Dehydrogenation ratio (GC)=57%. Colorless liquid. Z/E>99:1. Mixture: ¹H NMR (400 MHz, CDCl₃) δ 5.39 (t, J=4.7 Hz, 2H), 2.08 (dd, J=12.0, 6.4 Hz, 4H), 1.33 (m, 24H), 0.94 (t, J=6.8 Hz, 6H). ¹³C NMR (101 MHz, CDCl₃) δ 130.1, 32.3, 32.3, 30.2, 30.1, 30.1, 29.9, 29.8, 29.7, 29.7, 27.5, 23.0, 14.3. HRMS (APCI-MS ES+) [M−H]+, calculated for C₁₈H₃₇; 253.2896; found 253.2902.

cis-eicosenes (6): Corrected yield=695 mg (29%). Dehydrogenation ratio (GC)=32%. Colorless liquid. Z/E >99:1. Mixture: ¹H NMR (400 MHz, CDCl₃) δ 5.37 (t, J=4.6 Hz, 2H), 2.05 (dd, J=11.8, 6.2 Hz, 4H), 1.29 (s, 28H), 0.91 (t, J=6.8 Hz, 6H). ¹³C NMR (101 MHz, CDCl₃) δ 130.1, 32.2, 30.1, 30.0, 30.0, 29.7, 29.6, 27.5, 23.0, 14.3. HRMS (APCI-MS ES+) [M−H]+, calculated for C₁₈H₃₃; 249.2556; found 249.2582.

cis-1,n-hexadecadienes (7): Corrected yield=1197 mg (42%). Dehydrogenation ratio (GC)=63%. Colorless liquid. Z/E>99:1. Mixture: ¹H NMR (400 MHz, CDCl₃) δ 5.83 (ddt, J=16.9, 10.2, 6.7 Hz, 1H), 5.39 (dd, J=7.3, 3.7 Hz, 2H), 5.02 (d, J=17.1 Hz, 1H), 4.96 (dd, J=10.2, 1.0 Hz, 1H), 2.16-1.97 (m, 6H), 1.55-1.19 (m, 16H), 0.93 (t, J=6.8 Hz, 3H). ¹³C NMR (101 MHz, CDCl₃) 139.1, 130.2, 130.0, 129.9, 129.7, 114.4, 114.3, 114.3, 34.1, 34.1, 34.0, 32.2, 32.2, 32.1, 30.1, 30.0, 30.0, 30.0, 30.0, 29.9, 29.7, 29.6, 29.6, 29.5, 29.5, 29.4, 29.3, 29.3, 29.3, 29.2, 28.8, 27.5, 27.4, 27.3, 23.0, 23.0, 22.9, 14.3, 14.3. HRMS (APCI-MS ES+) [M−H]+, calculated for C₁₆H₃₁; 223.2426; found 223.2429.

cis-1,n-octadecadienes (8): Corrected yield=983 mg (46%). Dehydrogenation ratio (GC)=77%. Colorless liquid. Z/E>99:1. Mixture: ¹H NMR (400 MHz, CDCl₃) δ 5.83 (ddt, J=16.9, 10.2, 6.7 Hz, 1H), 5.48-5.23 (m, 2H), 5.02 (dd, J=17.1, 1.9 Hz, 1H), 4.98-4.92 (m, 1H), 2.13-1.98 (m, 6H), 1.49-1.22 (m, 18H), 0.92 (t, J=6.8 Hz, 3H). ¹³C NMR (101 MHz, CDCl₃) δ 139.2, 130.1, 129.9, 114.3, 34.0, 32.2, 30.0, 30.0, 29.9, 29.8, 29.6, 29.6, 29.4, 29.3, 29.2, 27.4, 27.4, 22.9, 14.3. HRMS (APCI-MS ES+) [M−H]+, calculated for C₁₈H₃₃; 249.2556; found 249.2582.

1-methoxy-cis-hexadecenes (9): Yield=888 mg (41%). Dehydrogenation ratio (GC)=89%. Colorless liquid. Z/E >99:1. Mixture: ¹H NMR (400 MHz, CDCl₃) δ 5.36-5.25 (m, 2H), 3.32 (t, J=6.6 Hz, 2H), 3.28 (s, 3H), 2.04-1.93 (m, 4H), 1. 57-1.47 (m, 2H), 1.36-1.17 (m, 20H), 0.85 (t, J=6.8 Hz, 3H). ¹³C NMR (101 MHz, CDCl₃) δ 130.0, 129.7, 72.9, 58.4, 32.0, 29.9, 29.8, 29.8, 29.7, 29.6, 29.4, 29.2, 27.3, 27.2, 26.2, 22.8, 14.1. HRMS (APCI-MS ES+) [M+H]+, calculated for C₁₇H₃₅O; 255.2666; found 255.2688.

1-chloro-cis-hexadecenes (10): Corrected yield=752 mg (34%). Dehydrogenation ratio (GC)=90%. Colorless liquid. Mixture: ¹H NMR (400 MHz, CDCl₃) δ 5.35 (m, 2H), 3.51 (t, J=6.8 Hz, 2H), 2.08-1.94 (m, 4H), 1.83-1.69 (m, 2H), 1.50-0.99 (m, 18H), 0.89 (t, J=6.7 Hz, 3H). ¹³C NMR (101 MHz, CDCl₃) δ 130.1, 129.5, 44.9, 32.7, 31.9, 29.8, 29.6, 29.4, 28.5, 27.2, 27.1, 26.8, 22.7, 14.1. HRMS (TOF-MS ES+) [M+H]+, calculated for C₁₆H₃₂Cl; 259.2191; found 259.2179.

1-chloro-cis-octadecenes (11): Corrected yield=783 mg (32%). Dehydrogenation ratio (GC)=48%. Colorless liquid. Z/E>99:1. Mixture: ¹H NMR (400 MHz, CDCl₃) δδ 5.41-5.28 (m, 2H), 3.51 (t, J=6.8 Hz, 2H), 2.03 (d, J=5.8 Hz, 2H), 1.82-1.69 (m, 2H), 1.50-1.15 (m, 20H), 0.90 (t, J=6.8 Hz, 3H). ¹³C NMR (101 MHz, CDCl₃) δ 130.1, 129.8, 45.0, 45.0, 32.9, 32.9, 32.1, 32.1, 31.8, 30.0, 29.9, 29.9, 29.9, 29.8, 29.8, 29.7, 29.6, 29.6, 29.6, 29.5, 29.4, 29.1, 29.1, 27.4, 27.3, 27.1, 27.1, 22.9, 22.8, 14.2. HRMS (APCI-MS ES+) [M+H]+, calculated for C₁₈H₃₆Cl; 287.2505; found 287.2536.

1-fluoro-cis-hexadecenes (12): Corrected yield=700 mg (34%). Dehydrogenation ratio (GC)=69%. Colorless liquid. Z/E>99:1. Mixture: ¹H NMR (400 MHz, CDCl₃) δ 5.40-5.26 (m, 2H), 3.51 (t, J=6.8 Hz, 2H), 2.10-1.95 (m, 4H), 1.83-1.69 (m, 2H), 1.50-1.18 (m, 18H), 0.89 (t, J=6.7 Hz, 3H). ¹³C NMR (101 MHz, CDCl₃) δ 130.2, 129.6, 45.1, 45.0, 32.8, 32.8, 32.8, 32.1, 32.1, 29.9, 29.9, 29.8, 29.8, 29.7, 29.7, 29.6, 29.5, 29.5, 29.1, 28.7, 27.4, 27.2, 27.1, 26.9, 22.8, 14.2. 19F NMR (377 MHz, CDCl₃) δ−211.1. HRMS (APCI-MS ES+) [M+H]+, calculated for C₁₆H₃₂F; 287.2505; found 287.2536.

1-fluoro-cis-octadecenes (13): Yield=851 mg (37%). Dehydrogenation ratio (GC)=72%. Colorless liquid. Z/E >99:1. Mixture: ¹H NMR (400 MHz, CDCl₃) δ 5.42-5.27 (m, 2H), 3.51 (t, J=6.8 Hz, 2H), 2.03 (d, J=5.8 Hz, 4H), 1.84-1.71 (m, 2H), 1.46-1.10 (m, 20H), 0.89 (t, J=6.8 Hz, 3H). ¹³C NMR (101 MHz, CDCl₃) δ 130.0, 129.8, 45.0, 32.8, 32.8, 32.1, 32.1, 29.9, 29.9, 29.9, 29.8, 29.8, 29.7, 29.6, 29.5, 29.5, 29.5, 29.3, 29.1, 27.4, 27.3, 27.1, 27.1, 22.8, 14.2. 19F NMR (377 MHz, CDCl₃) δ−211.3. HRMS (APCI-MS ES+) [M+H]+, calculated for C₁₈H₃₆F; 287.2505; found 287.2536.

Low Productivity (GC Yield <15% after 10 Days)

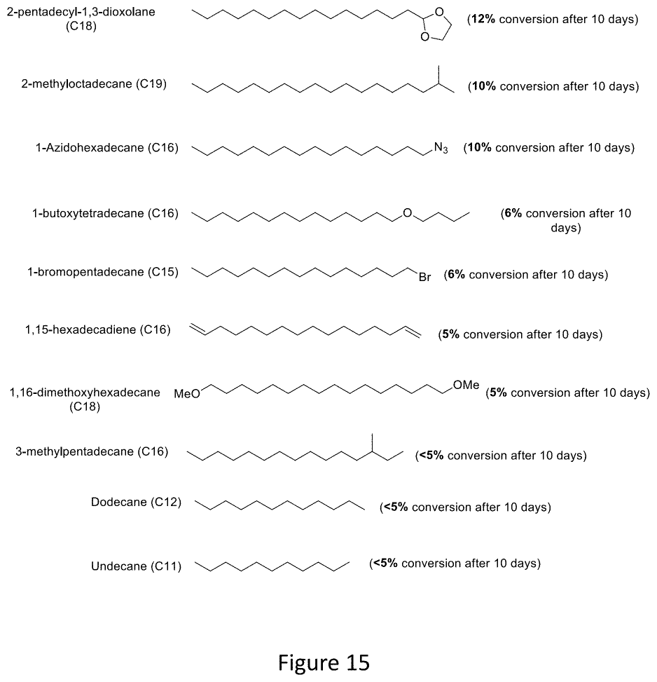

In the presence of the compounds presented in FIG. 15, the dehydrogenation reaction occurred but relatively slowly using the strain KSM-3-BM under standard reactional conditions.

No Productivity

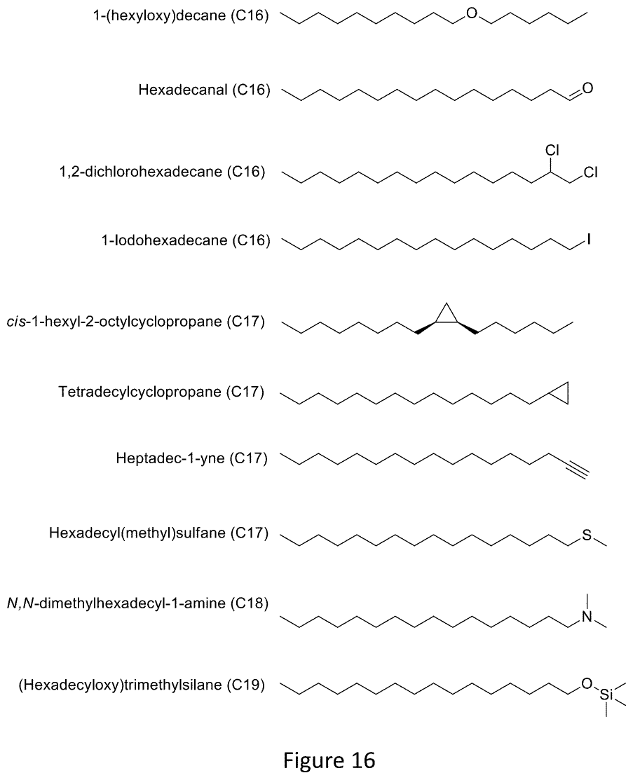

In the presence of the compounds presented in FIG. 16 no dehydrogenation reaction was observed using the strain KSM-3-BM under standard reactional conditions.

### Example 4

### Protocol for the Fermentation Process Using Isotopically Labelled Hexadecane

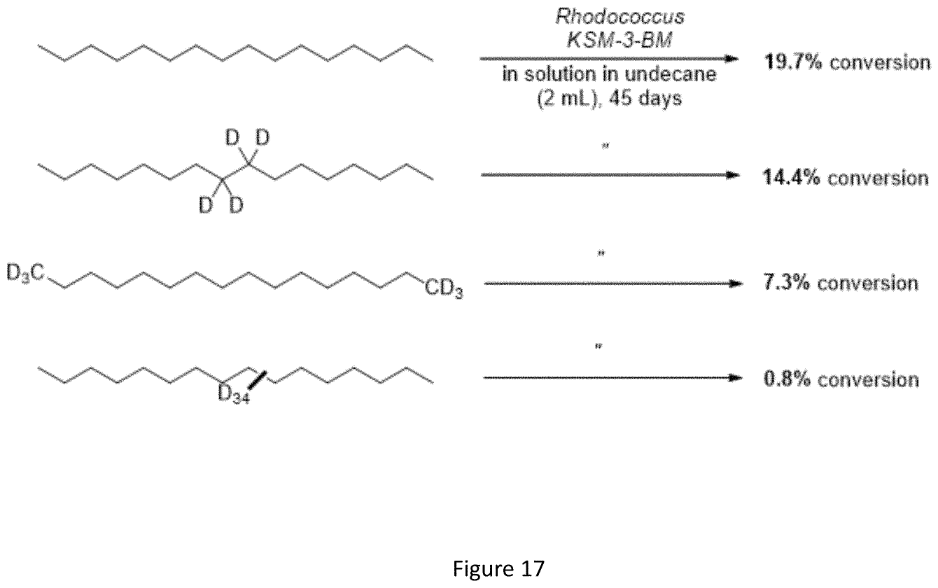

Similarly to the General procedure 2, a solution of the isotopically labelled hexadecane (1.9 mmol) dissolved in undecane (2 mL) was introduced in the fermentation process. The reaction was monitored by GC. After 45 days of reaction and following similar work-up protocol, these reactions afforded the results presented in FIG. 17.

### Example 5

### General Procedure for the Isomerization-Hydrozirconation Sequence

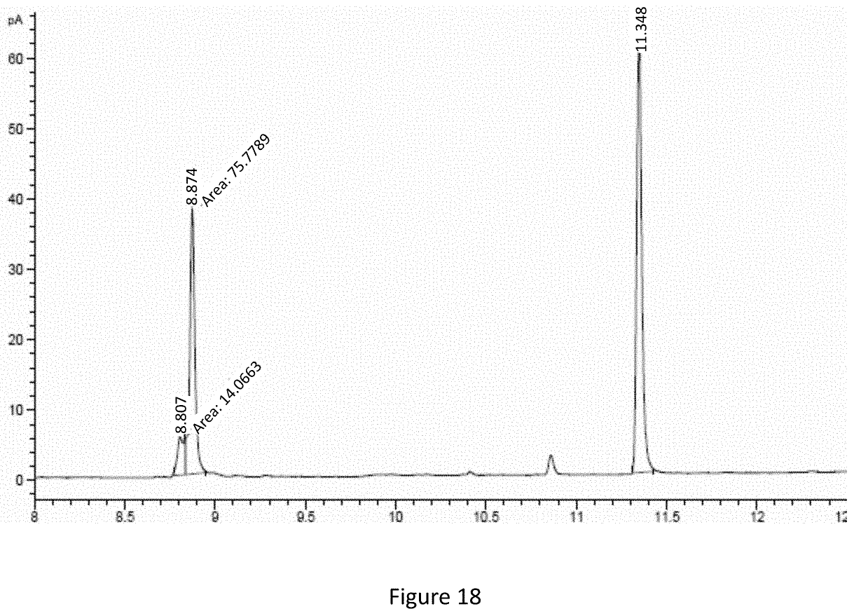

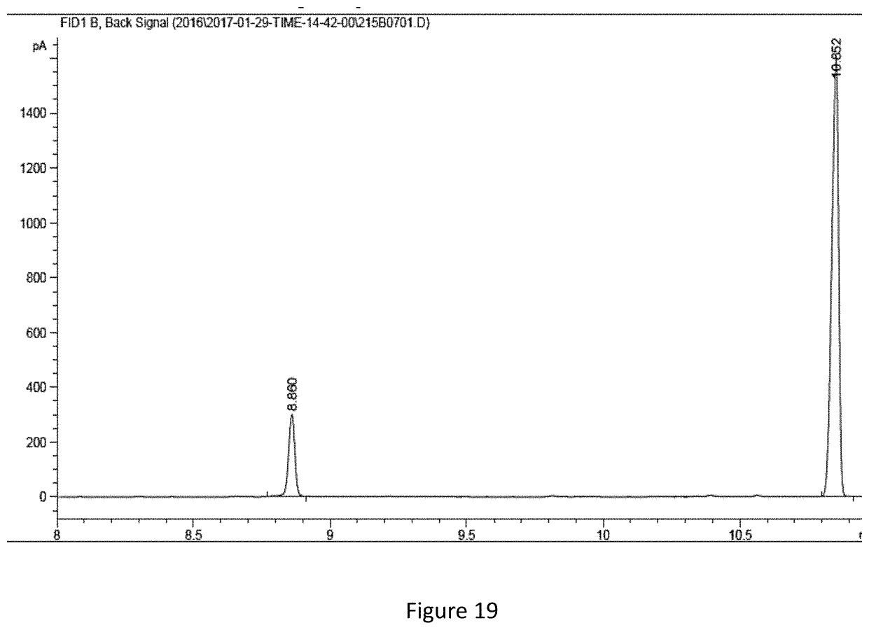

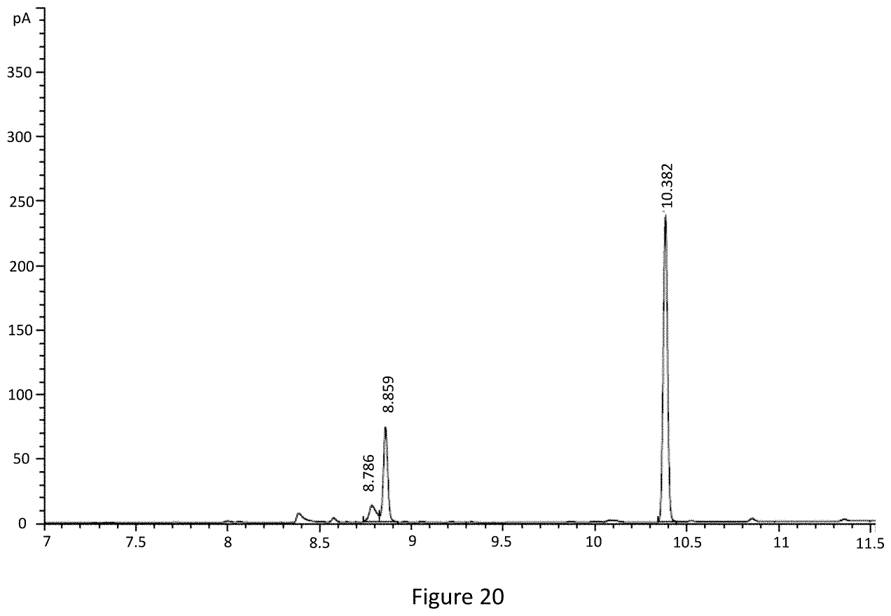

In a flamed-dried single neck round-bottom flask under Ar was added Cp₂ZrCl₂ (2.3 equiv, 1.25 mmol, 365 mg) in dry THF (10 mL) and this solution was stirred at RT for 10 min until complete dissolution. Then at RT, was slowly added Red-Al® (60 wt % in toluene, 1 equiv, 0.5 mmol, 0.140 mL, 3.5 M) (1 drop/3 s). The resulting reaction was stirred for 2h at RT. Then cis-hexadecenes (1) (83% GC purity, 1 equiv, 0.5 mmol, 113 mg) in THF (1 mL) was added to the reactional mixture at RT. The resulting mixture was stirred overnight (ca. 18 h) at 40° C., and one of the following exemplary procedures (A-C) were applied. The reactions were monitored by GC analyses of hydrolysed aliquots (FIGS. 18-20).

Procedure A: Halogenolysis (NCS, Br₂ or I₂)

Then mixture was cooled down to 0° C. and the electrophile (3 equiv, 1.5 mmol, dissolved in 1 mL of dry THF), was slowly added. The mixture was stirred for 3h at RT. Then, the solution was quenched with a saturated solution of sodium thiosulfate (5 mL) and extracted with Et₂O (3×10 mL). The crude solution was evaporated under vacuum and the mixture was filtrated on a short pad of silica with hexane to yield the desired product. Yields were evaluated by GC analyses on the crude residue either using dodecane or hexadecane as internal standards.

1-Iodohexadecane (C₁₆H₃₃I): Performing the electrophilic trapping with 12 (3 equiv, 1.5 mmol, 379 mg) afforded 1-iodohexadecane in 76% GC yield as a mixture with remaining hexadecane. All related spectroscopic data matched with commercially available pure 1-iodohexadecane.

1-bromohexadecane (C₁₆H₃₃Br): Performing the electrophilic trapping with Br₂ (3 equiv, 1.5 mmol, 238 mg) afforded 1-bromohexadecane in 95% GC yield as a mixture with remaining hexadecane. All related spectroscopic data matched with commercially available pure 1-bromohexadecane.

1-chlorohexadecane (C₁₆H₃₃Cl): Performing the electrophilic trapping with N-chlorosuccinimide (3 equiv, 1.5 mmol, 200 mg) afforded 1-chlorohexadecane in 86% GC yield as a mixture with remaining hexadecane. All related spectroscopic data matched with commercially available pure 1-chlorohexadecane.

Procedure B: oxidation (tBuOOH)

Then the solution was cooled down to 0° C. and tBuOOH (3 equiv, 1.5 mmol, 0.3 mL, C=5 M in decane) was slowly added. The mixture was stirred 2h at RT (TLC monitoring). Then, water (5 mL) was added and the resultant mixture was extracted with Et₂O (3×10 mL). The combined organic layers were washed with brine (10 mL), dried (Na₂SO₄), and filtered. The volatiles were removed under reduced pressure and the residue obtained was purified by flash chromatography (hexane/Et₂O, 7:3) affording 72 mg of 1-hexadecanol as a white waxy solid, yield=59%. All related spectroscopic data matched with commercially available pure 1-hexadecanol.

Procedure C: Copper-Catalyzed Allylation Reactions

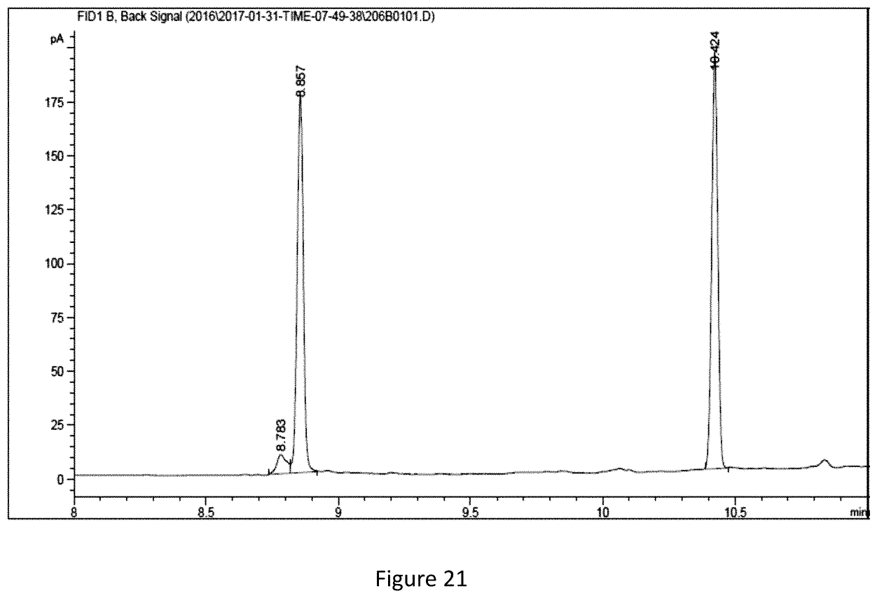

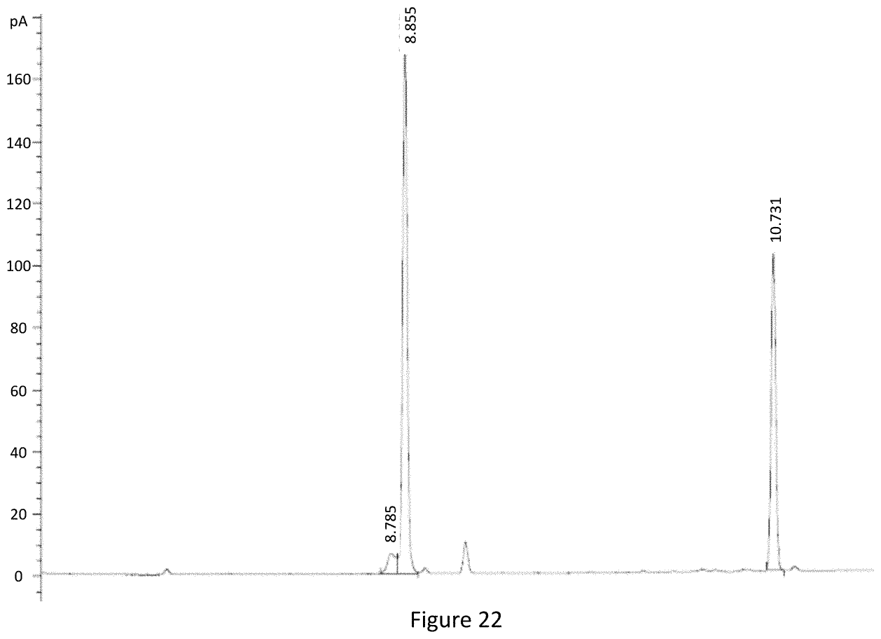

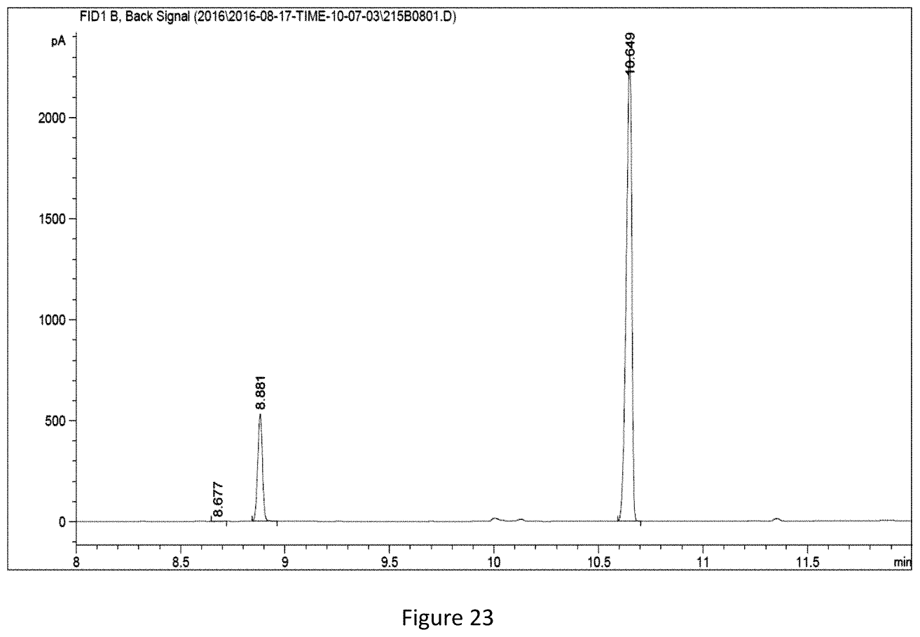

Then CuI (0.2 equiv, 20 mg, 0.1 mmol) and flame-dried LiCl (0.4 equiv, 9 mg, 0.2 mmol) brought into solution in THF (3 mL) were added at 0° C., and stirred 30 min at this temperature. Then, the electrophile (3 equiv, 1.5 mmol), dissolved in THF (1 mL), was slowly added at 0° C. The resulting solution was stirred for 24h at RT. Then, the solution was quenched with saturated solution of ammonium chloride (5 mL) and extracted with Et₂O (3×10 mL). The crude solution was dried and evaporated under vacuum. Yields were evaluated by GC analyses on the crude residue using dodecane as internal standard (FIGS. 21-22).

1-nonadecene (C₁₉H₃₆): Using freshly distilled allyl bromide (3 equiv, 1.5 mmol, 180 mg, 0.13 mL) after transmetallation with CuI.2LiCl afforded 1-nonadecene in 60% GC yield as a mixture with hexadecane (FIG. 21).

Nonadec-1,2-d

iene (C₁₉H₃₆): Using freshly distilled allyl bromide (3 equiv, 1.5 mmol, 180 mg, 0.13 mL) after transmetallation with CuI.2LiCl afforded 1-nonadecene in 60% GC yield as a mixture with hexadecane (FIG. 22).

Ethyl-2-methylenenonadecanoate:

Using ethyl 2-(bromomethyl)acrylate (3 equiv, 1.5 mmol, 290 mg) after transmetalation with CuI.LiCl to afford 81 mg of ethyl-2-methylenenonadecanoate as white waxy solid in 48% isolated yield after purification on flash chromatography (hexane/Et₂O, 95:5). ¹H NMR (400 MHz, CDCl₃) δ 6.13-6.11 (m, 1H), 5.50 (dd, J=2.8, 1.4 Hz, 1H), 4.20 (q, J=7.1 Hz, 2H), 2.32-2.24 (m, 2H), 1.44 (d, J=8.4 Hz, 2H), 1.33-1.21 (m, 31H), 0.87 (t, J=6.8 Hz, 3H). ¹³C NMR (101 MHz, CDCl₃) δ 167.6, 141.3, 124.3, 60.7, 32.1, 32.0, 29.9, 29.8, 29.8, 29.7, 29.6, 29.5, 29.4, 28.6, 22.8, 14.4, 14.3.

Procedure D: Electrophilic Amination

Following the hydrozirconation step, the solution was cooled down to room temperature and NH₂OSO₃H or MeNH₂OSO₃H (2.5 equiv, 1.25 mmol) was added in one portion. The resulting suspension was vigorously stirred for 3 h at 50° C. After completion, aqueous 1 M NaOH (5 mL) was added. The mixture was transferred to a separatory funnel and extracted with Et₂O (3×10 mL). The combined organic phases were washed with brine, dried over Na₂SO₄, and concentrated in vacuo. The crude residue was purified by column chromatography to afford the desired products. 1-Hexadecylamine (C₁₆H₃₃N): White solid (78.4 mg, 65%). ¹H NMR (400 MHz, CDCl₃) δ 2.63 (s, 2H), 2.23 (s, 2H), 1.41-1.36 (m, 1H), 1.21-1.19 (m, 26H), 0.81 (t, J=6.8 Hz, 3H). 13C NMR (101 MHz, CDCl₃) δ 42.0, 33.4, 31.9, 29.7, 29.6, 29.5, 29.3, 26.9, 22.7, 14.1. The NMR spectra matched those previously reported in the literature.

Procedure E: Cyanation

Following the hydrozirconation step, TMSCN (1.5 mol, 3 equiv) was added and the reaction mixture was heated at 60° C. for 24 h. The solution was then cooled down to 0° C. and a solution of iodine (1.5 mmol, 3 equiv) in THF (1 mL) was added. The reactional mixture was stirred for 3h at RT. Then, the solution was quenched with saturated solution of sodium thiosulfate (5 mL) and extracted with Et₂O (3×10 mL). The combined organic phases were washed with brine, dried over Na₂SO₄, and concentrated in vacuo. The crude residue was purified by column chromatography to afford the desired product. 1-heptadecanitrile: ¹H NMR (400 MHz, CDCl₃) δ 2.31 (t, J=7.1 Hz, 2H), 1.68-1.58 (m, 2H), 1.44-1.39 (m, 2H), 1.24 (m, 24H), 0.86 (t, J=6.8 Hz, 1H). ¹³C NMR (101 MHz, CDCl₃) δ 119.7, 31.8, 29.6, 29.6, 29.5, 29.5, 29.5, 29.5, 29.4, 29.2, 29.2, 28.6, 28.5, 25.3, 22.6, 17.0, 14.0. The NMR spectra matched those previously reported in the literature.

### Example 6

### General Procedure for the Isomerization-Hydroboration Sequence

In a sealed tube under Ar, [Rh(C₂H₄)Cl]₂ (5 mol %), triphenylphosphine (6 mol %), and the resulting olefin 1 (0.5 mmol) were dissolved in dry DCM (5 mL). HBpin (1.5 equiv.) was then introduced at RT. The reactional mixture was sealed then heated at 60° C. for 18 h. After being cooled down to 0° C., the suspension was diluted with MeOH (5 mL) and a solution of NaOH (3M, 1 mL) and H₂O₂ (30%, 1 mL) were added. The reactional mixture was let to stir at room temperature. The reaction was then neutralized with a solution of aqueous saturated NH₄Cl (5 mL) and the aqueous phase was extracted with EtOAc. The combined organic phases were dried over anhydrous MgSO₄ and concentrated under vacuum. The desired product was obtained after concentration under reduced pressure. A flash column chromatography (25% Et₂O/hexane) yielded pure 1-hexadecanol in a 30% overall yield.

### Example 7

### General Procedure for the Isomerization-Hydrosilylation Sequence

In a sealed tube under Ar, H₂PtCl₆.6H₂O (5 mol %), the resulting olefin 1 (0.5 mmol) and HSiCl₃ (1 mmol) were successively introduced. The neat reactional mixture was then heated at 130° C. for 18 h. After being cooled down to 0° C., the reaction was quenched with different nucleophiles (1 mmol) at that temperature then let to stir at room temperature for additional 6 h. Following a work-up protocol, the crude mixture was filtered through a PTFE HPLC filter (0.4 μm) to remove the remaining platinum particles and extensively washed with hexane. The desired product was obtained after concentration under reduced pressure.

Hexadecyltrimethylsilane: Using methylmagnesium bromide (3M in Et₂O, 6 equiv.) for 4h as nucleophile. Yield=98% (+20% remaining hexadecane). Mixture: ¹H NMR (400 MHz, CDCl₃) δ 1.27 (s, 28H), 0.89 (t, J=6.8 Hz, 3H), 0.49 (m 2H), −0.02 (s, 9H). ¹³C NMR (101 MHz, CDCl₃) δ 33.8, 32.1, 29.9, 29.9, 29.6, 24.1, 22.9, 16.9, 14.3, −1.5. All characterization data matched those reported in the literature for hexadecyltrimethylsilane.

Hexadecyltrivinylsilane: Using vinylmagnesium bromide (1M in THF, 6 equiv.), 60° C. for 4h. Yield=85% (+33% hexadecane). Mixture: ¹H NMR (400 MHz, CDCl₃) δ 6.31-5.61 (m, 9H), 1.26-1.10 (m, 28H), 0.81 (t, J=6.8 Hz, 3H), 0.69-0.48 (m, 2H). 13C NMR (101 MHz, CDCl₃) δ 135.6, 135.4, 135.0, 134.9, 134.8, 32.1, 29.9, 29.8, 29.5, 22.9, 14.3, 1.2. ²⁹Si NMR (80 MHz, CDCl₃) δ−21.9. HRMS (APCI-MS ES+) [M+H]+, calculated for C₂₂H₄₃Si; 335.3134; found 335.3112.

Triethoxy(hexadecyl)silane: Using ethanol (6 equiv.) and pyridine (3 equiv.) in THF (1 mL) at 60° C. for 6h. Yield=61% (+40% remaining hexadecane). All characterization data matched with commercially available triethoxy(hexadecyl)silane.

### Example 8

### General Procedure for the Recycling of the Microorganism

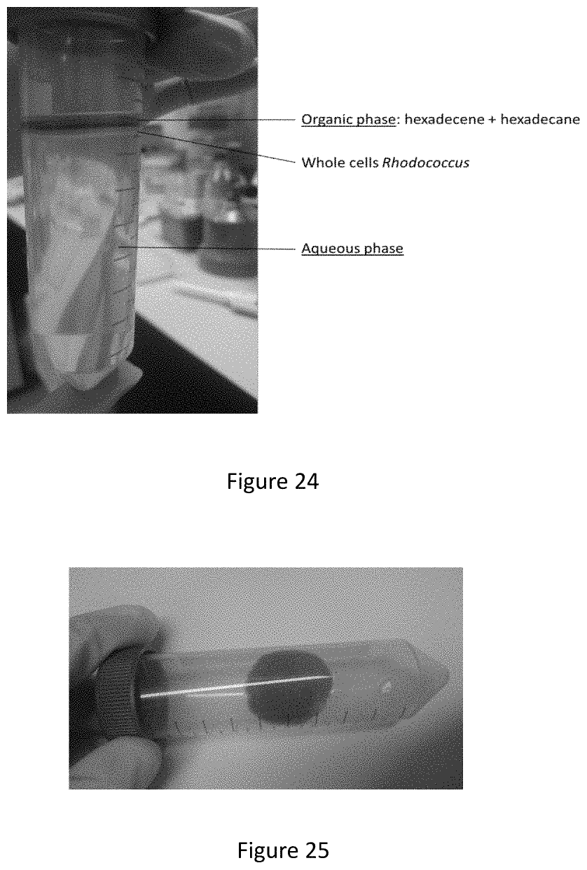

Following the general procedure of Example 2, using hexadecane as model substrate, the biphasic suspension was transferred into a falcon and centrifuged at 4800 rpm for 10 min until the appearance of three visible phases/layers (FIG. 24).

The middle phase containing the bacteria strain was recovered by carefully removing both organic and aqueous phases with a sterile pipette. The obtained pellet was washed with sterile saline (5 mL), dissolved and centrifuged (4700 rpm, 5 min). After removal of the saline phase, the resulting orange pellet (FIG. 25) was then dissolved in the sterile medium and transferred into a sterile Erlenmeyer following the general procedure of Example 2 previously described.

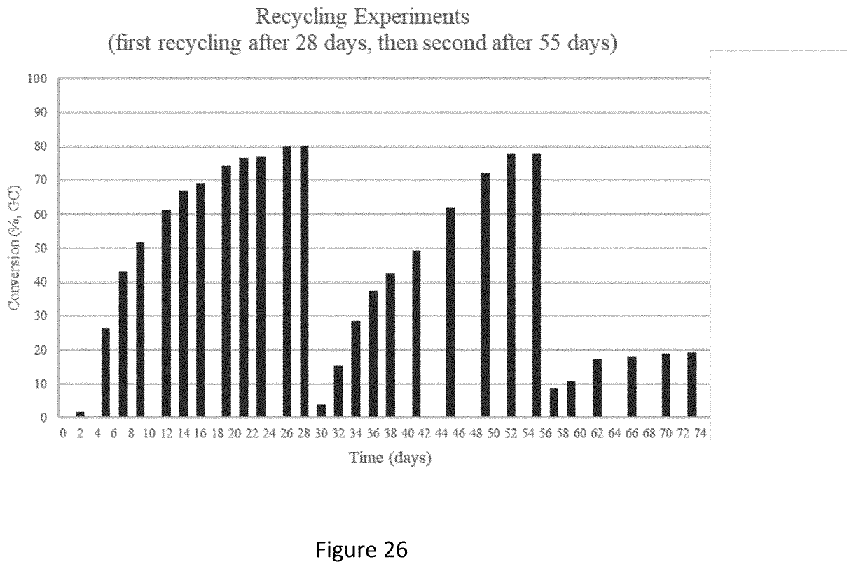

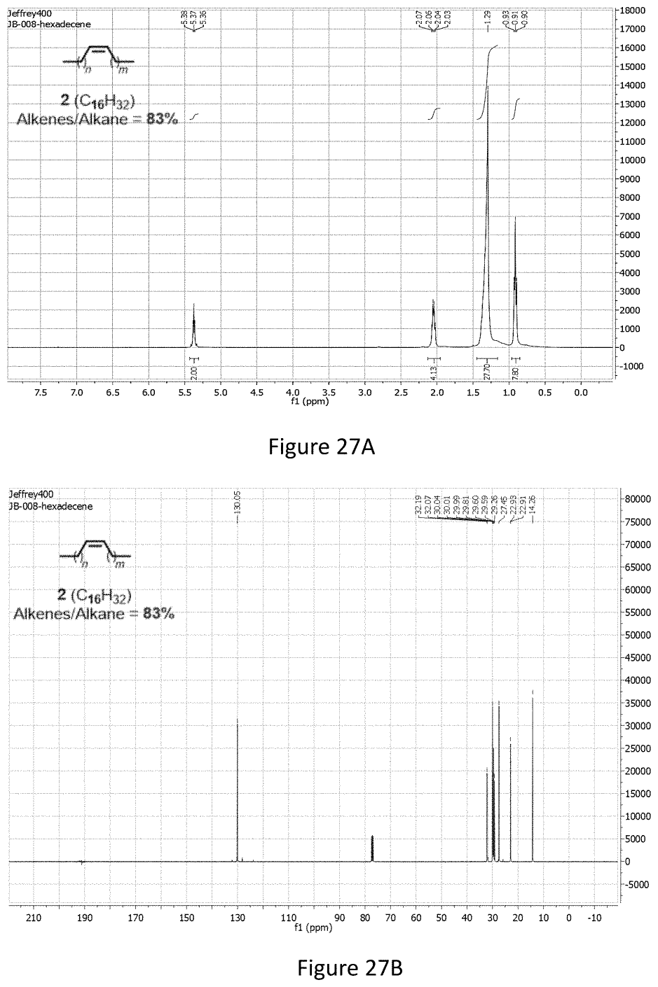

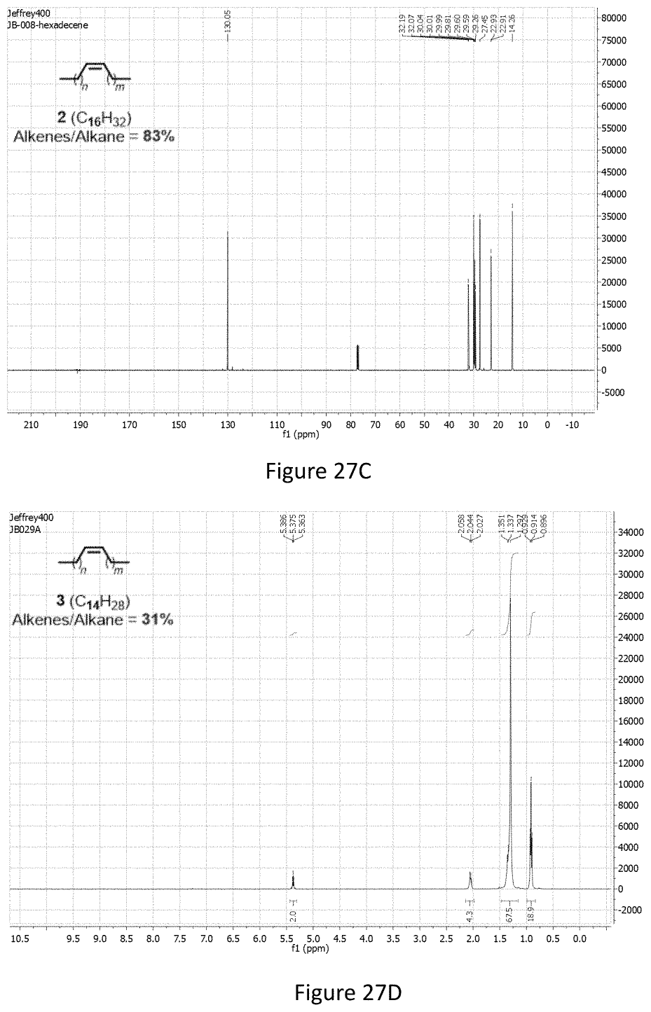

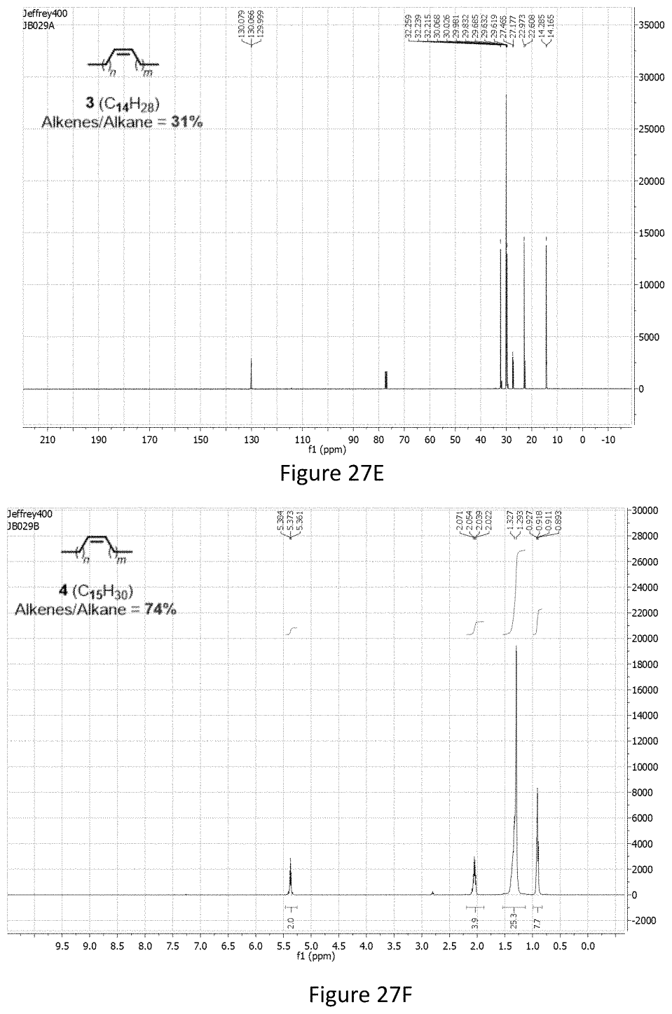

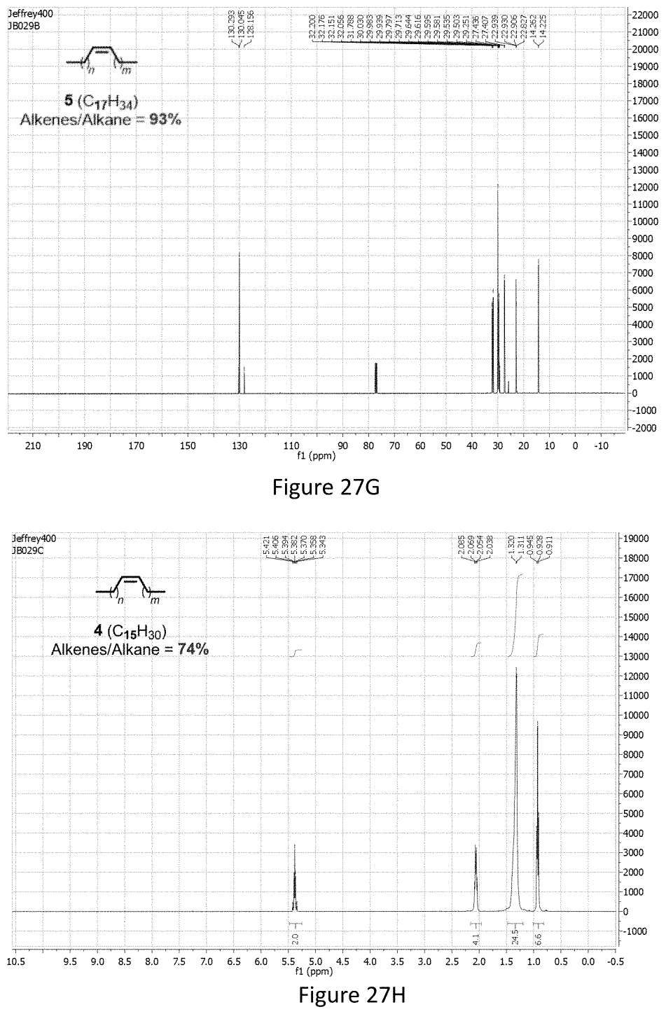

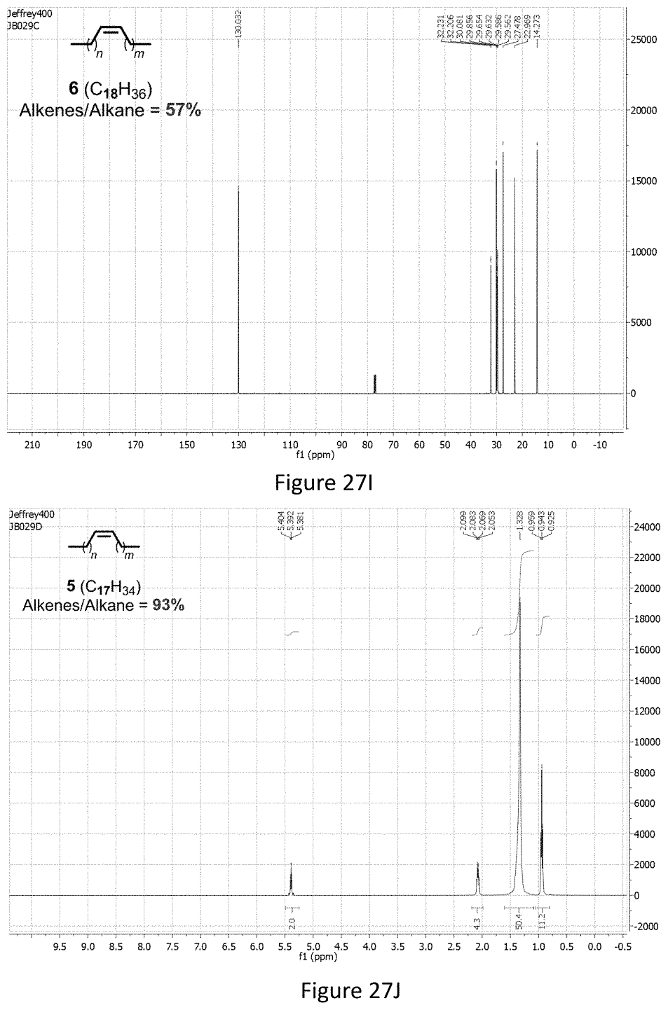

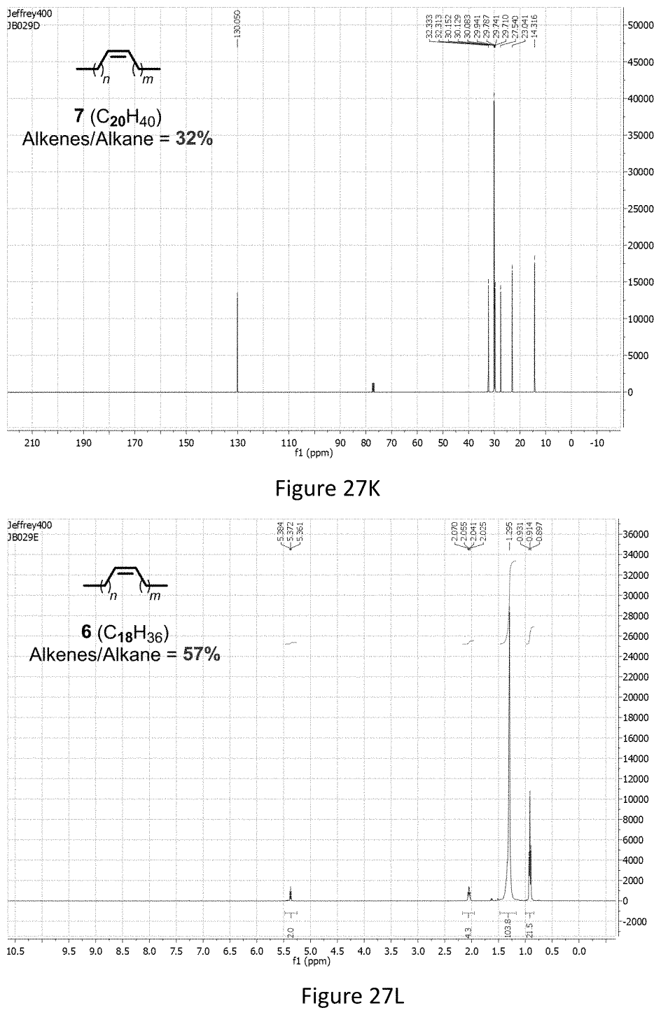

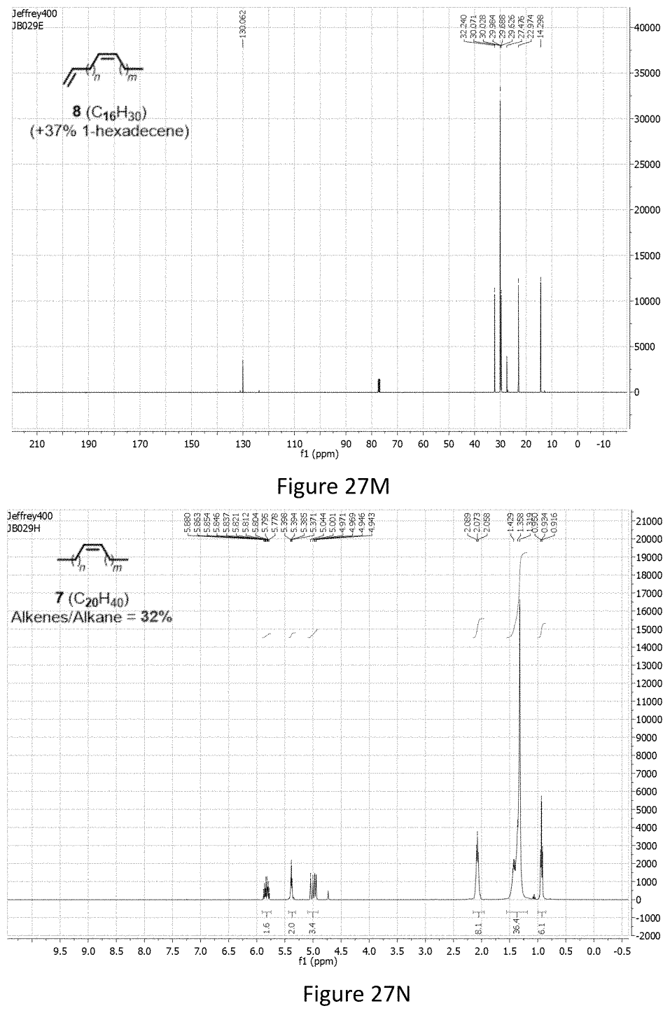

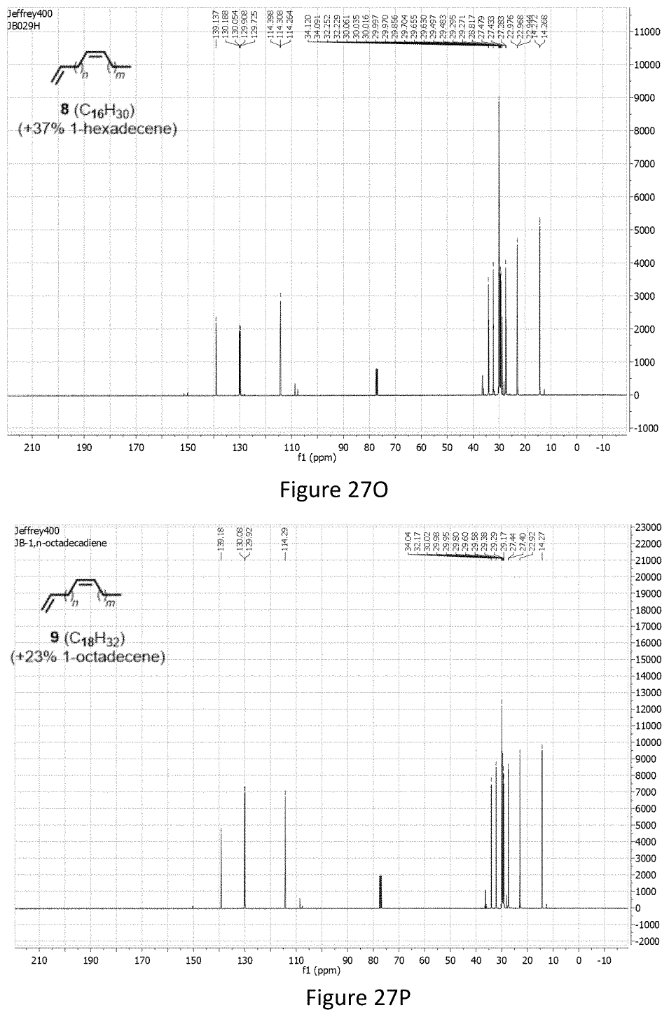

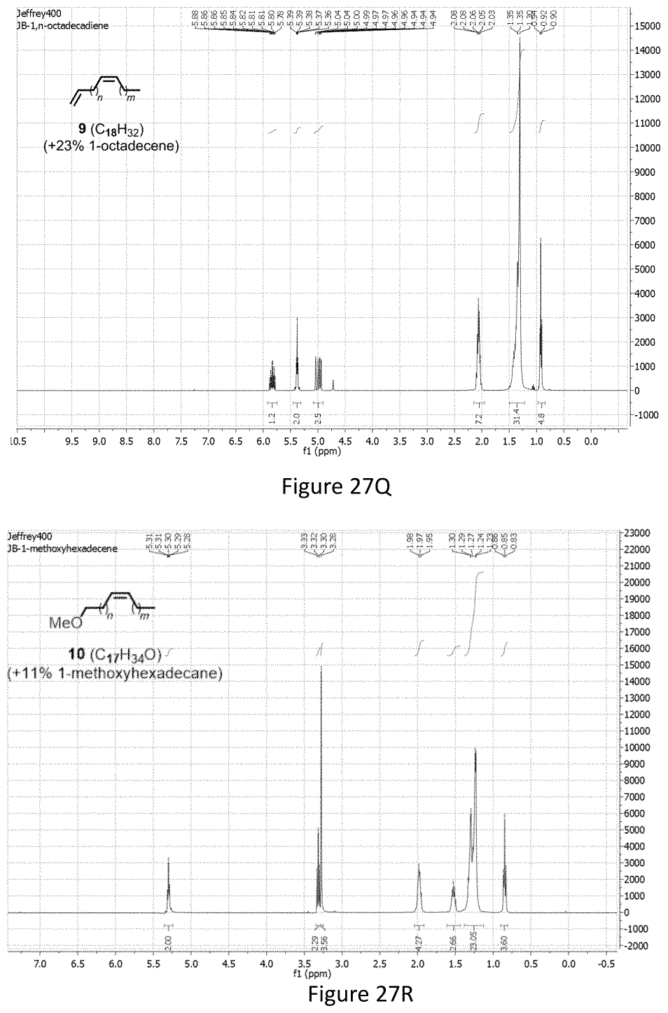

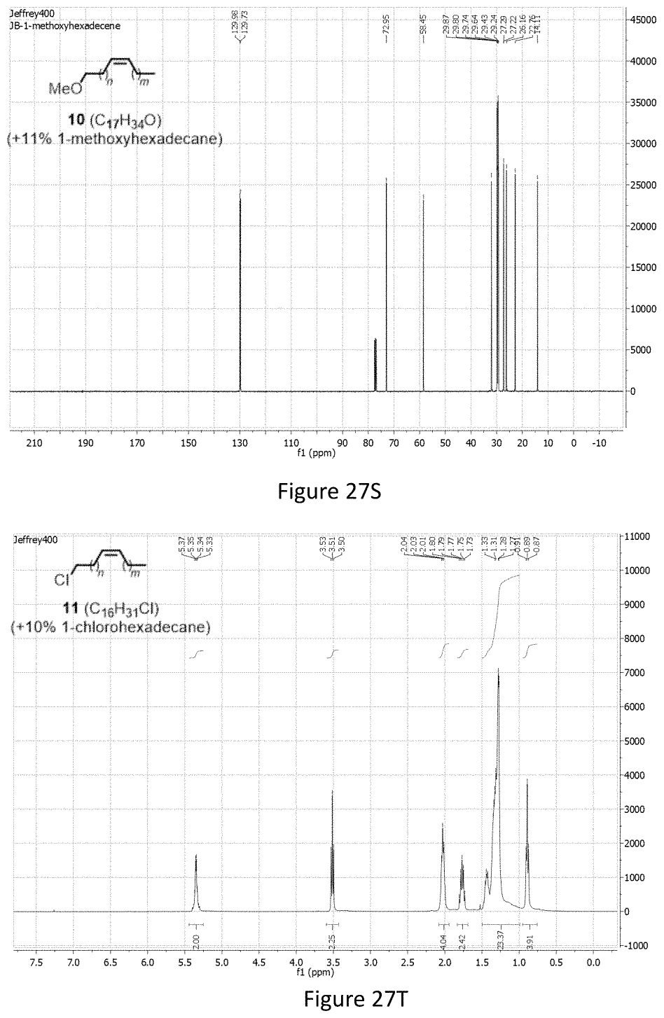

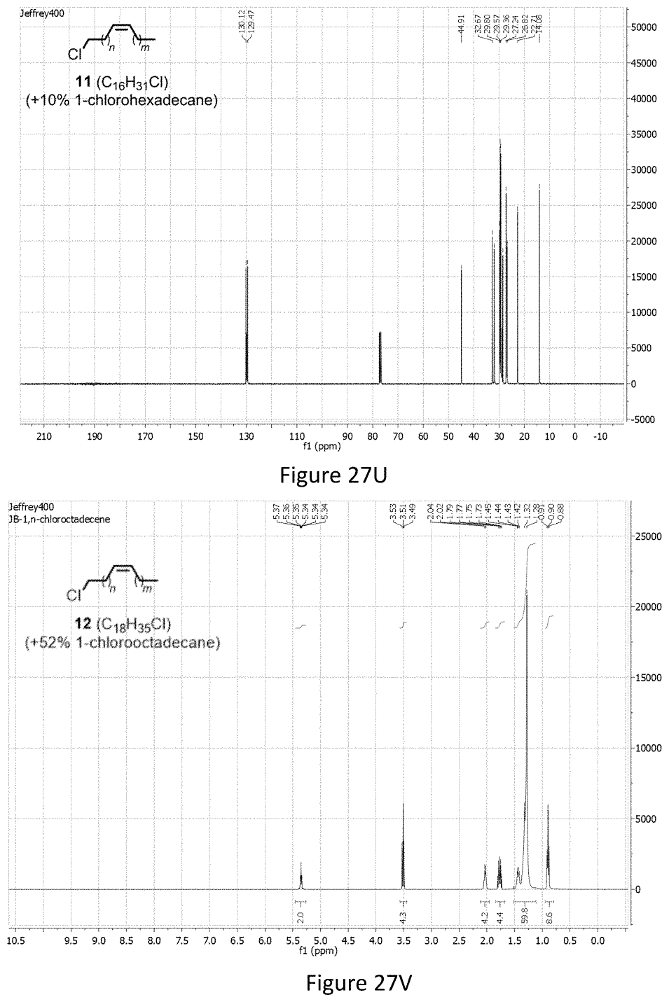

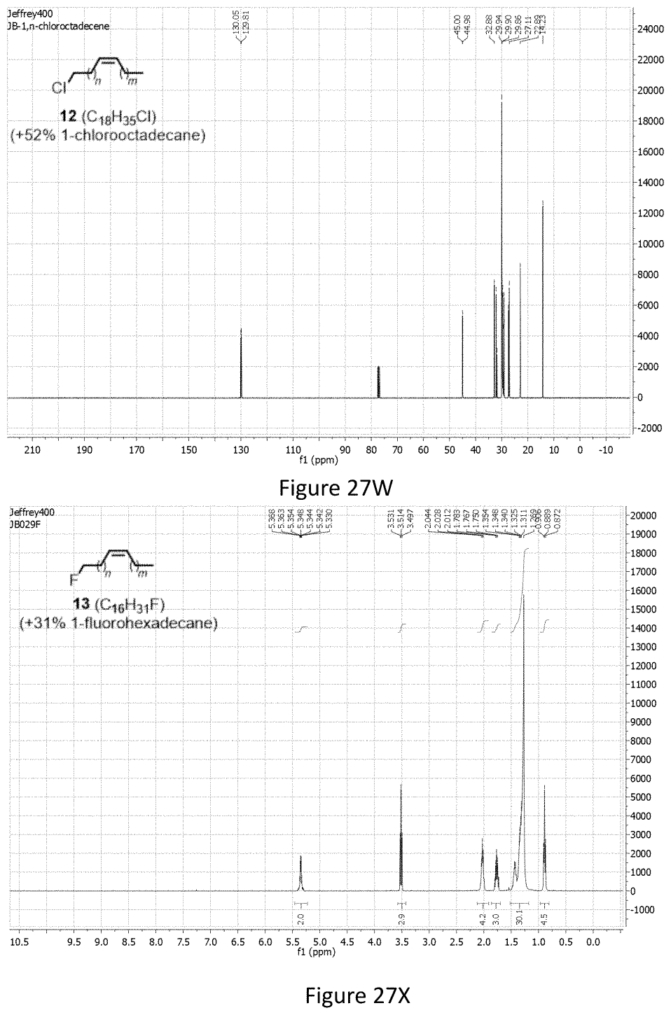

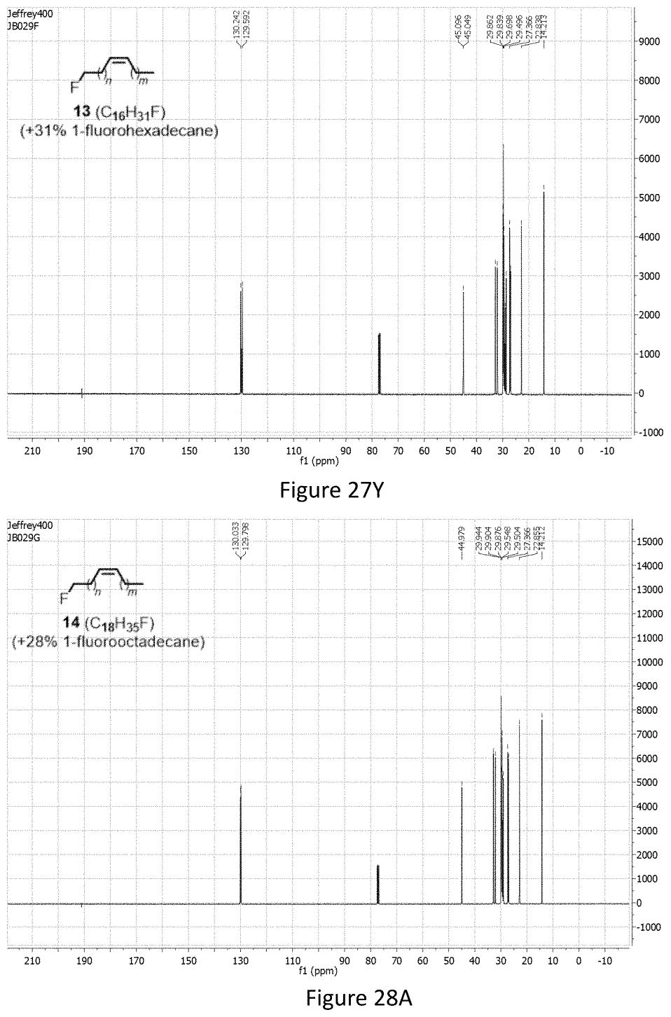

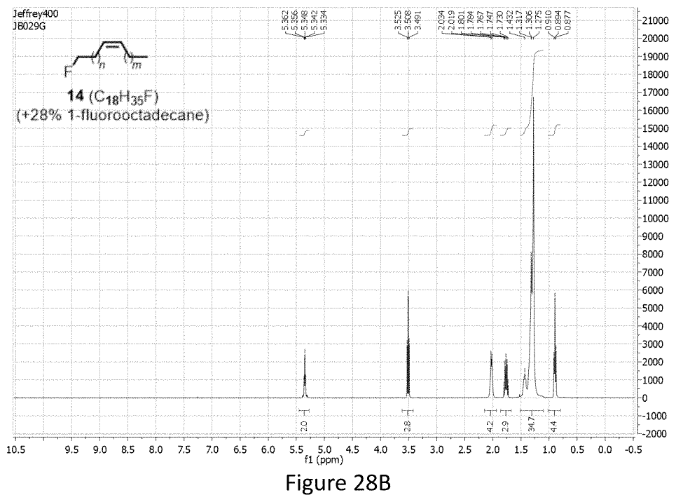

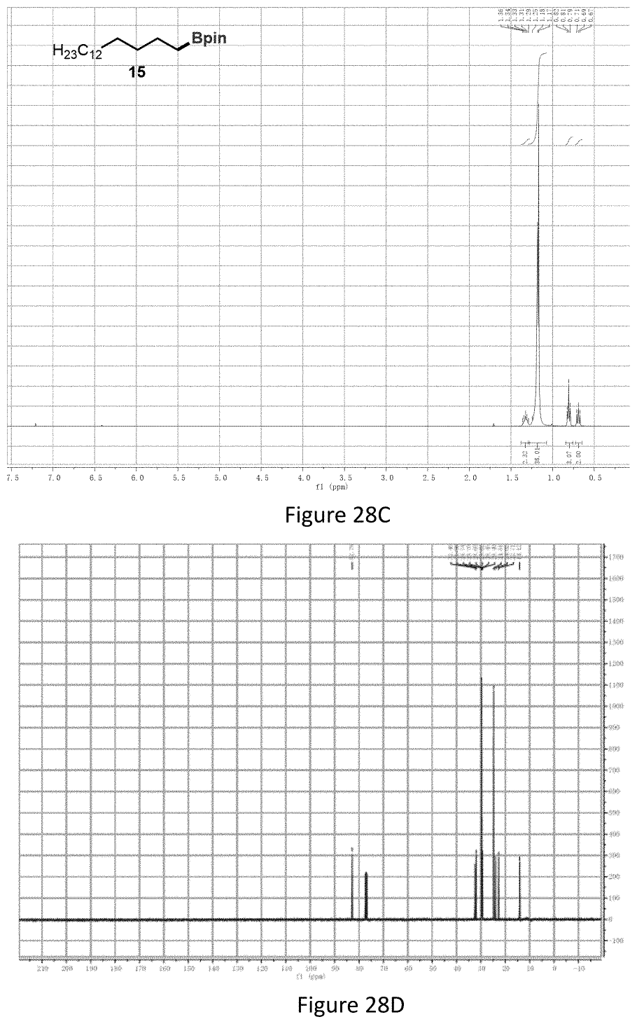

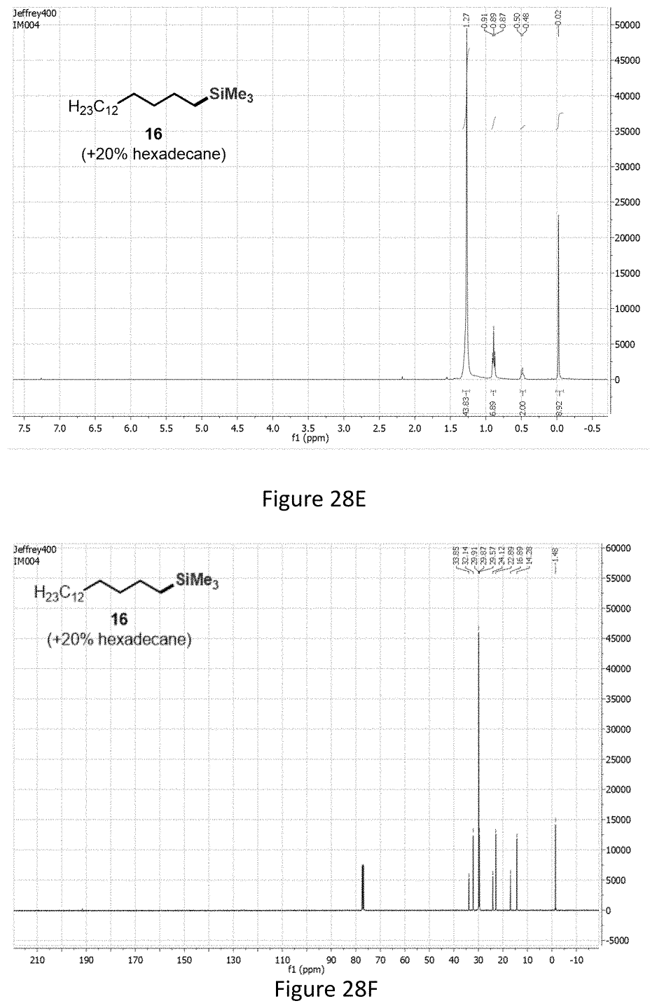

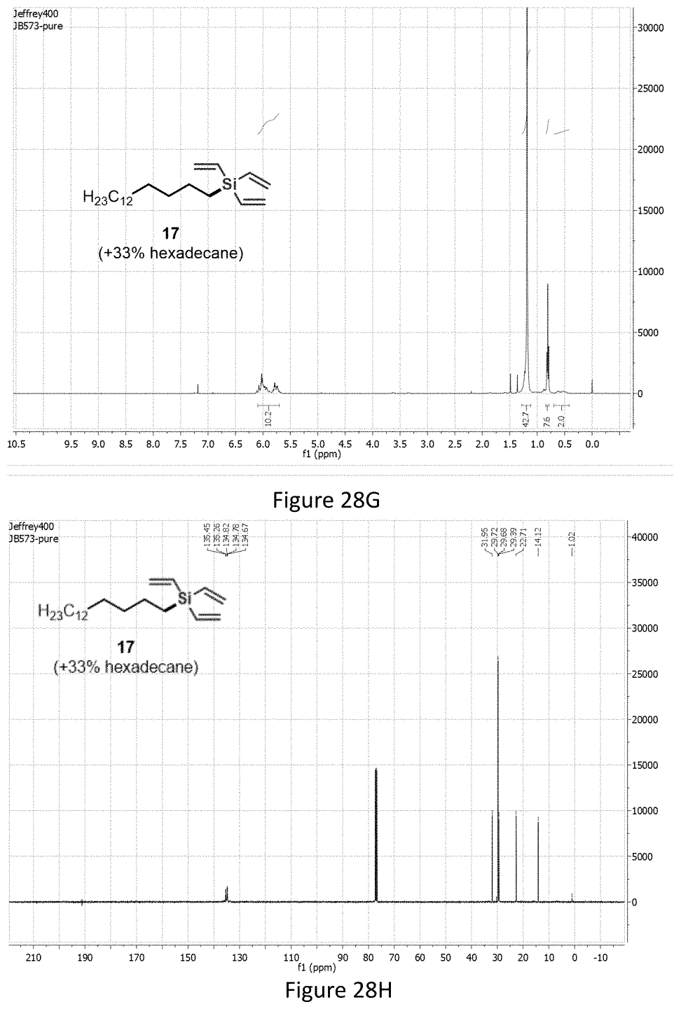

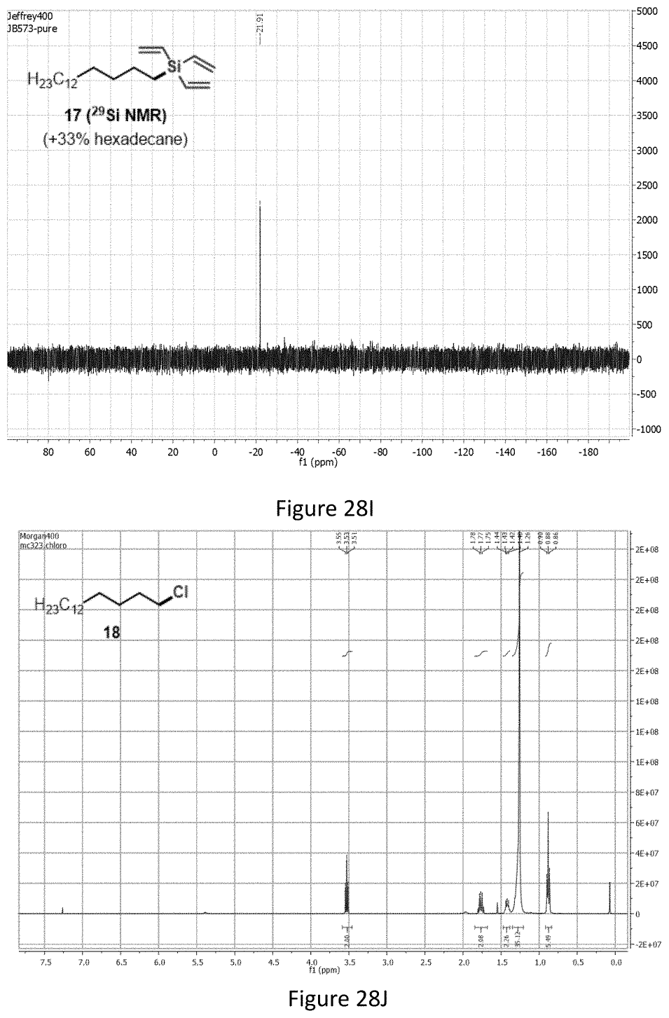

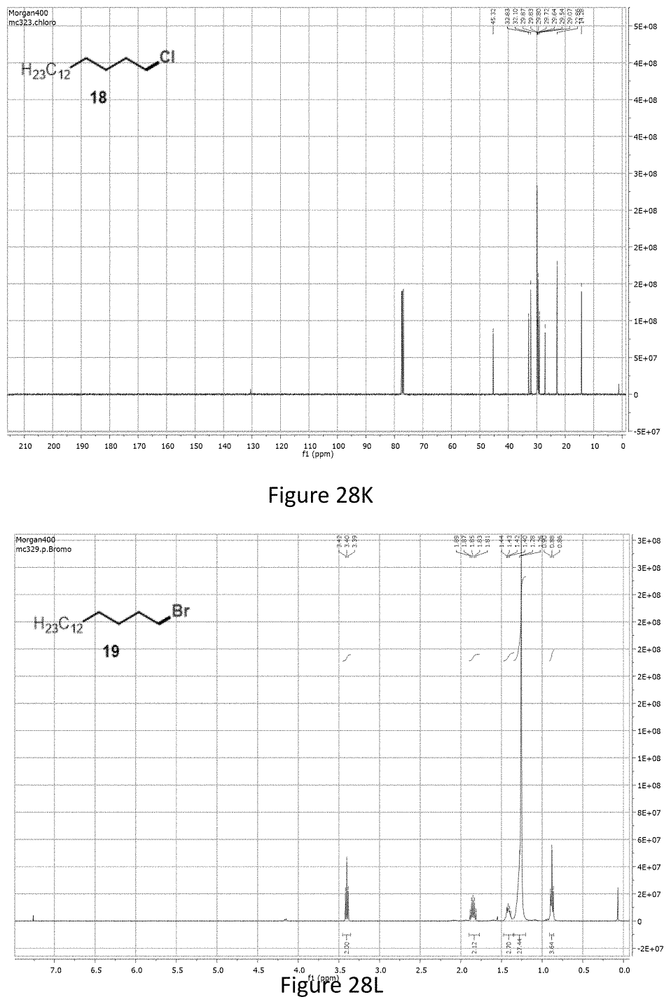

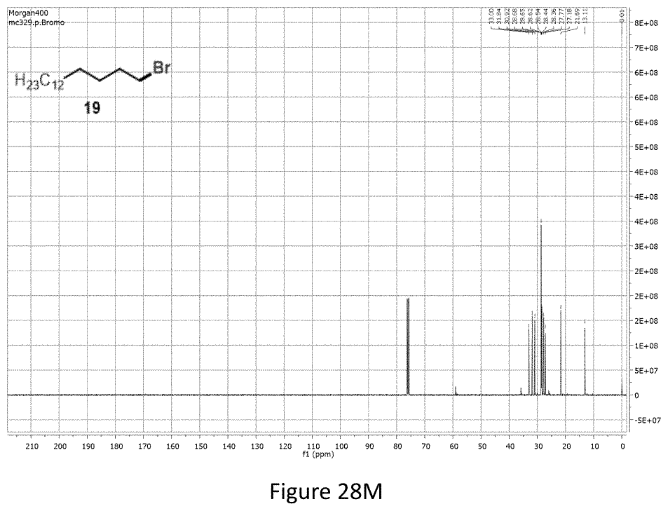

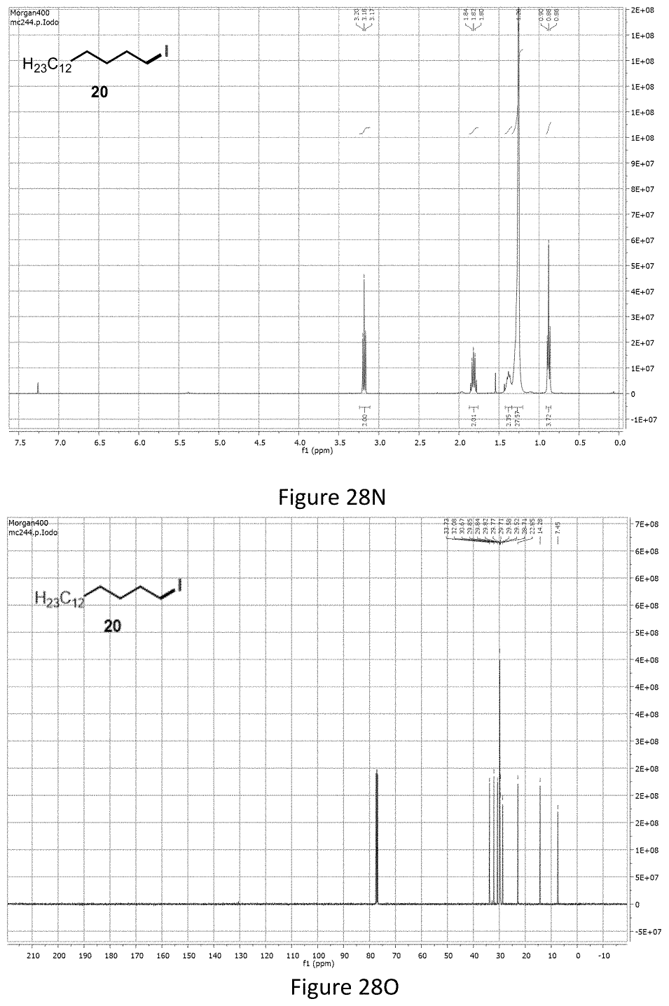

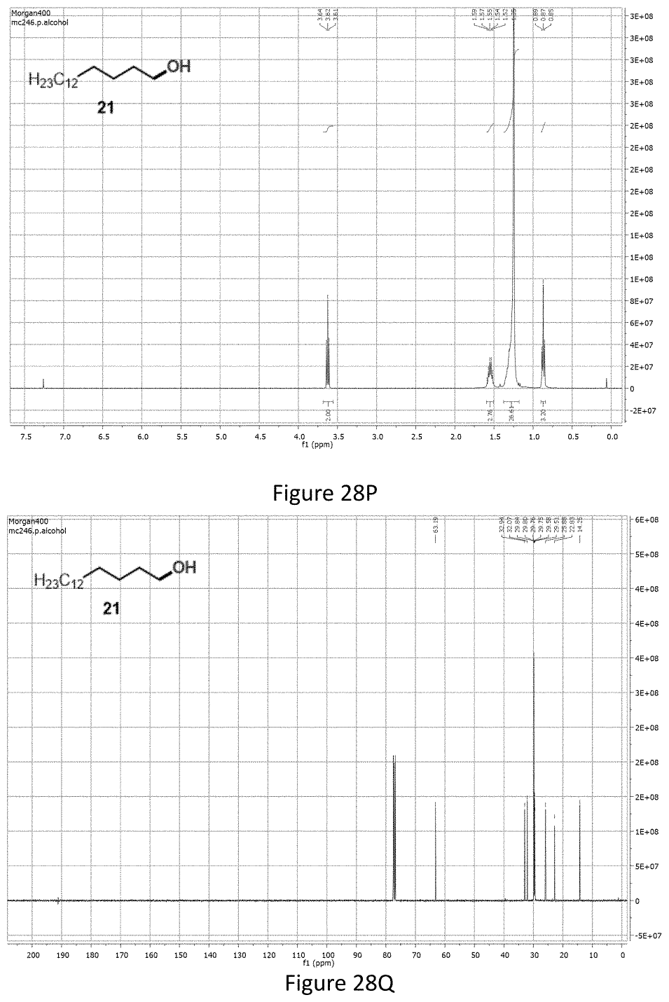

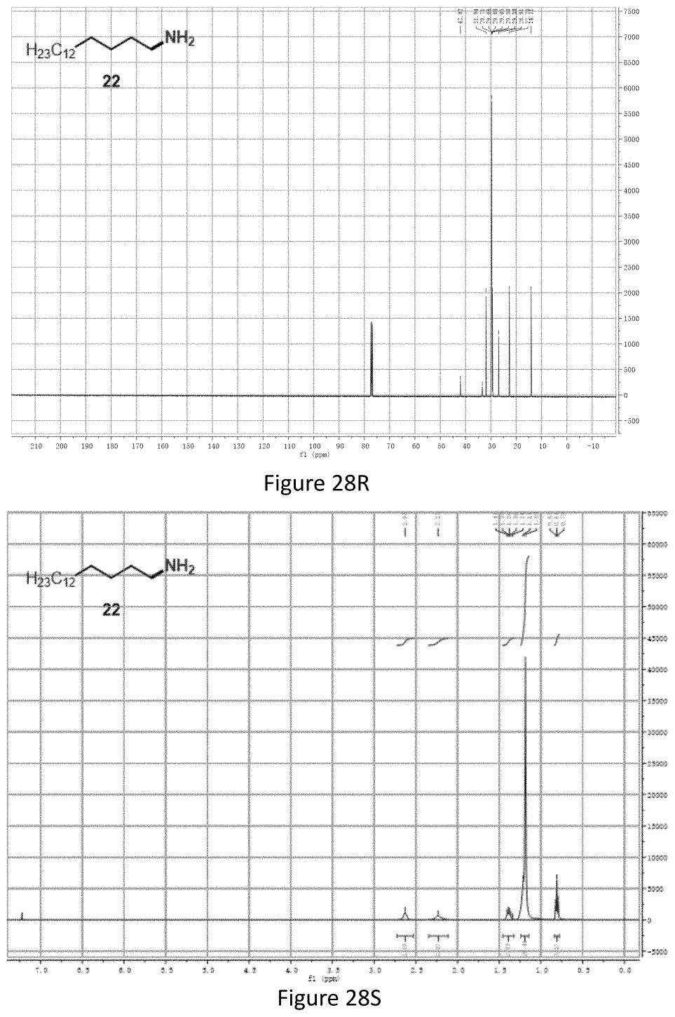

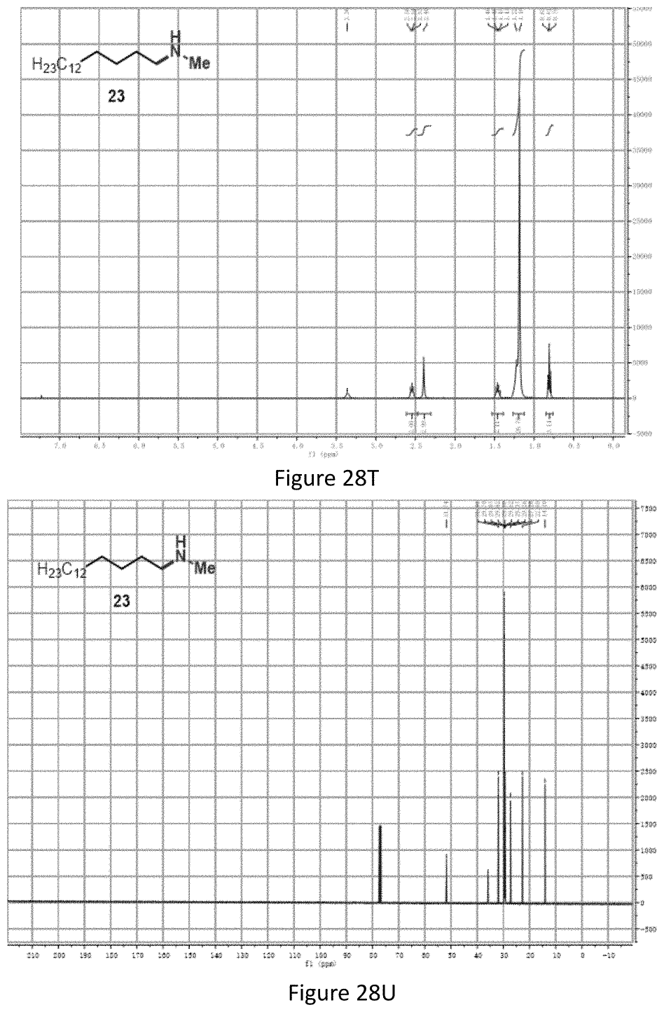

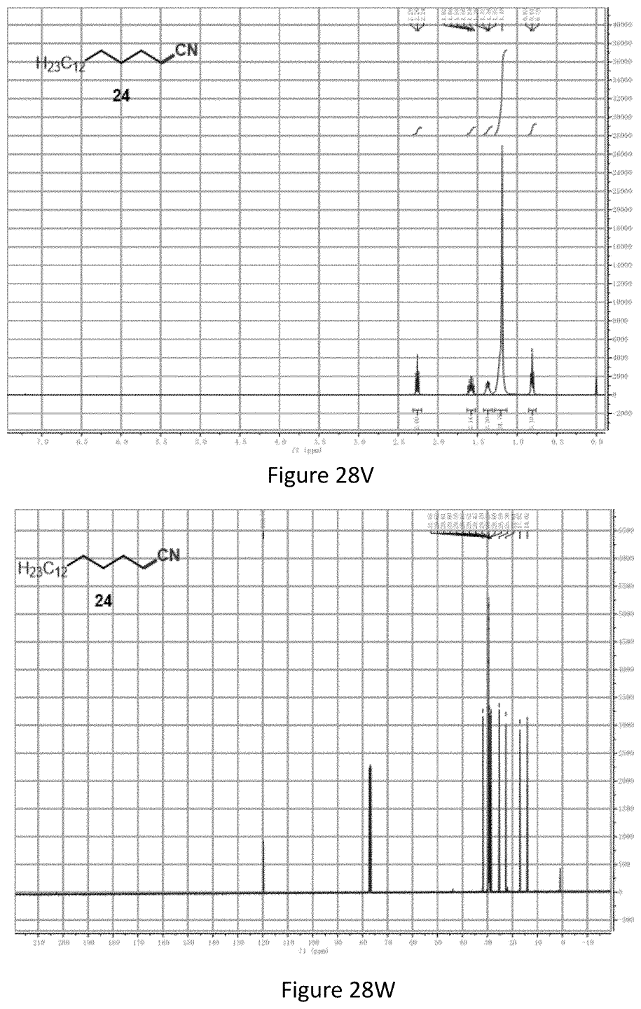

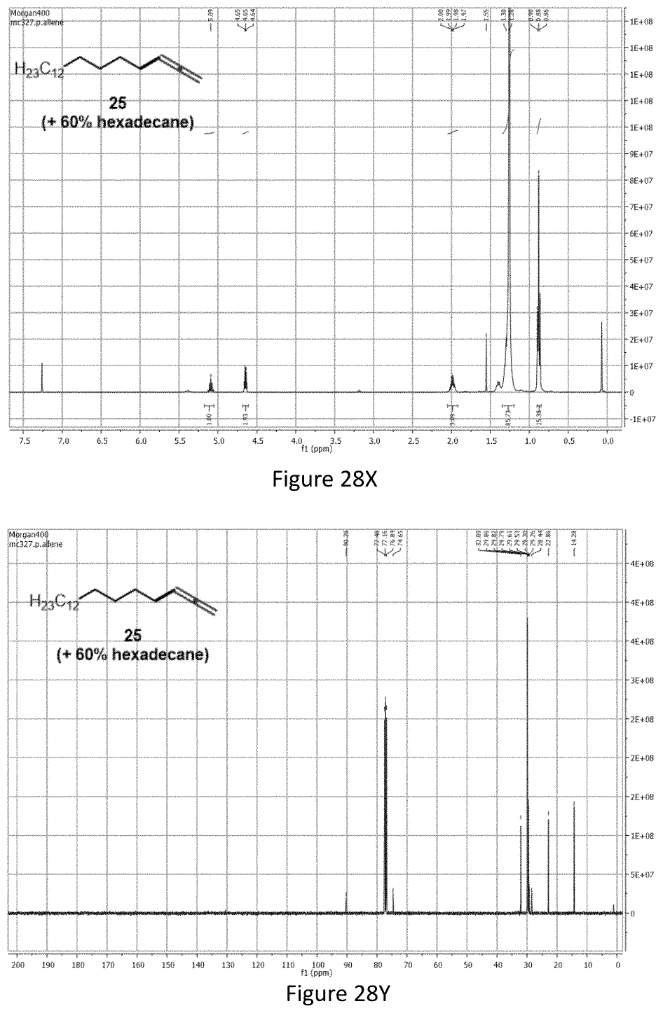

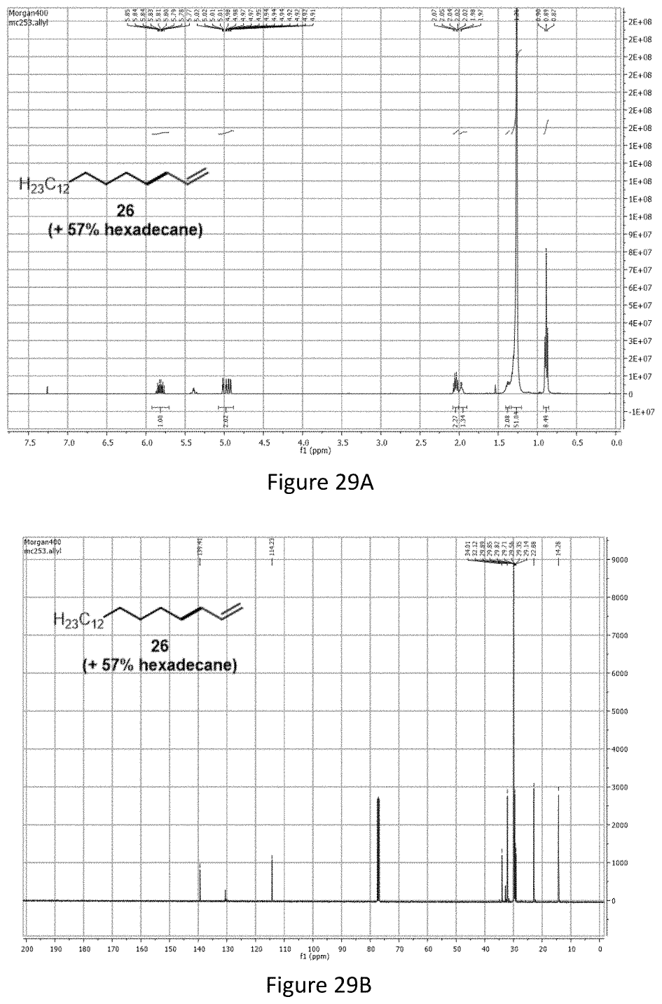

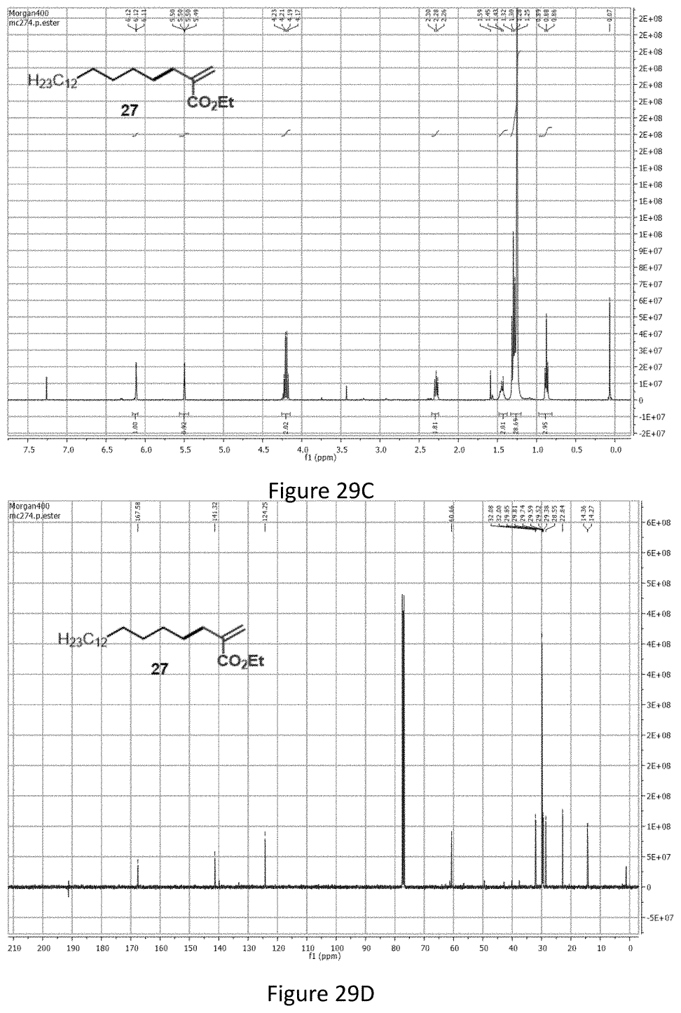

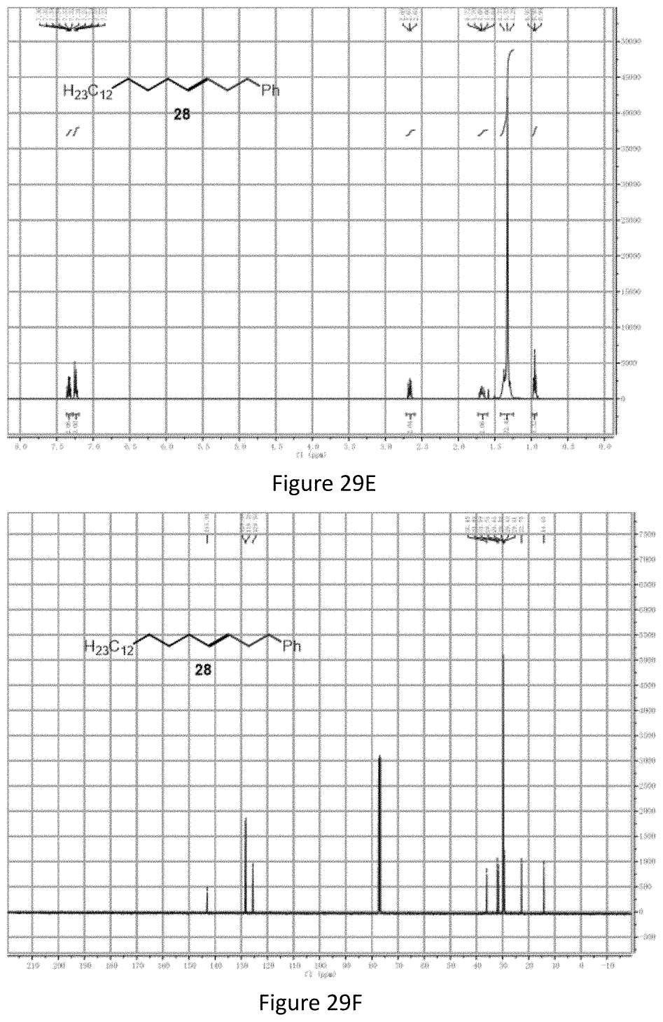

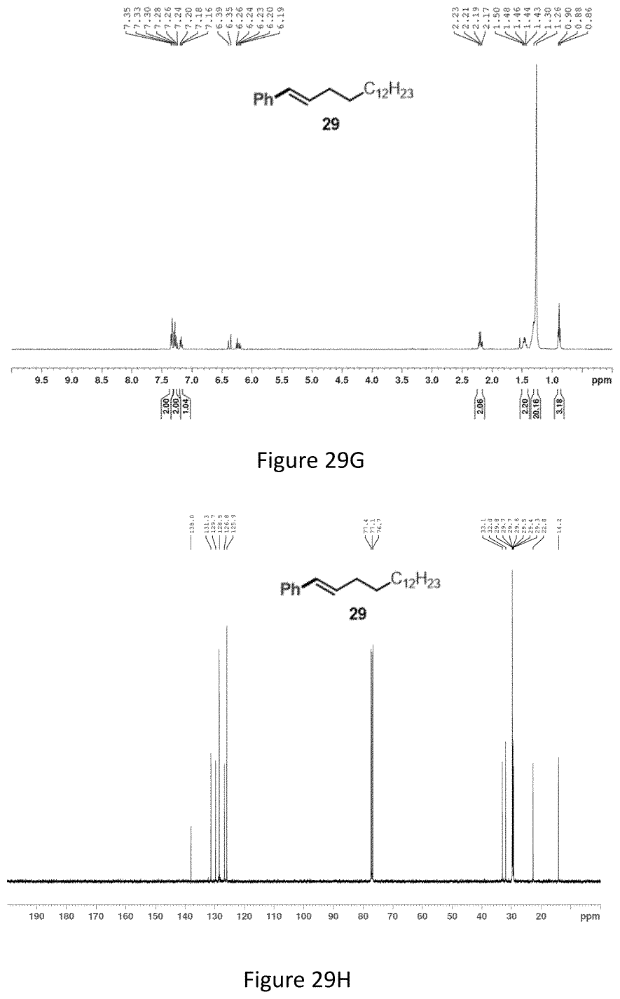

This process was repeated twice (around 74 days). Samples (0.25 mL) were taken every 2 or 3 days for GC analyses (FIG. 26).

### Example 9

### General Procedure for the Isomerization-Hydroboration Sequence

In a flamed tube with stir bar under Argon (Ar), the cobalt precatalyst (1 mol %) was added, the tube was evaporated and refilled with Ar for 3 times. Anhydrous toluene (2 mL) was added and the mixture was cooled to −35° C. Then, MeLi (2 mol %, 1.6 M in hexane) was added dropwise and stirred at RT for 5 min. Then, the mixture of cis-hexadecenes 2 (83% GC purity, 1 equiv, 0.5 mmol, 113 mg) and pinacolborane (0.5 mmol) were successively added. The reaction mixture was stirred at RT for 24 h. The reaction was quenched by exposing the solution to air. The resulting solution was concentrated in vacuum and the crude residue was purified by column chromatography to afford the desired product 2-hexadecyl-4,4,5,5-tetramethyl-1,3,2-dioxaborolane in 60% yield. ¹H NMR (400 MHz, CDCl₃) δ 1.36-1.28 (m, 2H), 1.125-1.17 (m, 38H), 0.81 (t, J=6.8 Hz, 3H), 0.69 (t, J=7.7 Hz, 2H). ¹³C NMR (101 MHz, CDCl₃) δ 82.7, 32.4, 31.9, 29.7, 29.7, 29.7, 29.6, 29.4, 29.4, 24.8, 24.0, 22.7, 14.1. HRMS (TOF-MS ES⁺) [M+14]⁺, calculated for C₂₂H₄₆BO₂; 353.3591; found 353.3590.

### Example 10

### Procedure for the Isomerization-Hydroarylation Sequence

To a flamed-dried tube was added NiBr₂ (5 mol %), 2-(6-methylpyridin-2-yl)-4,5-dihydrooxazole (6 mol %), KF (1.25 mmol, 2.5 equiv) and the vessel was purged 3 times with argon. Anhydrous DMF (1 mL) was added and the resulting suspension was stirred at RT for 10 min. Then, (EtO)₂MeSiH (1.25 mmol, 2.5 equiv) was added dropwise and stirred at RT for 5 min. After that, cis-hexadecenes 2 (83% GC purity, 1 equiv, 0.5 mmol, 113 mg) and (3-iodopropyl)benzene (1.25 mmol, 2.5 equiv) were added. The reactional mixture was stirred at room temperature for 24 h. After completion, water (20 mL) was added to the reaction mixture, transferred into a separatory funnel and extracted with Et₂O (3×10 mL). The combined organic phases were washed with brine, dried over Na₂SO₄, and concentrated in vacuo. The crude residue was purified by column chromatography to afford the desired product as 45% yield. 1-nonadecylbenzene: ¹H NMR (400 MHz, CDCl₃) δ 7.41-7.35 (m, 2H), 7.31-7.26 (m, 3H), 2.73-2.68 (m, 2H), 1.72 (dt, J=15.0, 7.4 Hz, 2H), 1.52-1.26 (m, 32H), 1.00 (t, J=6.8 Hz, 3H). ¹³C NMR (101 MHz, CDCl₃) δ 142.9, 128.3, 128.1, 125.5, 36.0, 31.9, 31.5, 29.7, 29.6, 29.6, 29.5, 29.3, 29.3, 22.7, 14.1. HRMS (TOF-MS ES+) [M+H]+, calculated for C₂₅H₄₄; 344.3438; found 344.3442.

### Example 11

### Procedure for the [Ru]-Catalyzed Isomerization/[Ni]Cross-Coupling Sequence of Alkenyl Methyl Ether

To a flame-dried Schlenk flask equipped with a reflux condenser, RuHCl(CO)(PPh₃)₃ (5 mol %, 0.022 mmol, 21 mg) and a solution of 1-methoxy-cis-hexadecenes (9) (synthetized as described in Example 3) (0.433 mmol, 110 mg) in THF (1.5 mL) were added under an inert atmosphere of argon. The mixture was stirred vigorously at 90° C. for 48 h, and then allowed to cool to ambient temperature. Then followed by to a second flame-dried Schlenk flask equipped with a septum and stirrer bar was added NiCl₂(PPh₃)₂ (10 mol %, 0.043 mmol, 28 mg) and the flask was purged with argon. Toluene (1.5 mL), and phenylmagnesium bromide (1.1 M in ether, 0.866 mmol, 0.8 mL) were introduced by a syringe, and then the reaction mixture was stirred at ambient temperature for 15 min. After this time, the solution of resulting vinyl methyl ether intermediate in the first Schlenk flask was added dropwise to the second reaction mixture via syringe under argon, and then the second reaction flask was sealed with a new septum. The resulting mixture was heated at 90° C. for 16 h. Then the reaction was cooled to ambient temperature, quenched with an aqueous saturated solution of NH₄Cl and extracted with Et₂O (3 mL×3). The combined organic phase was filtered through a pad of silica gel with copious washing by Et₂O and concentrated. The residue was purified by column chromatography on silica gel (eluent, hexane/toluene=20/1) to give a mixture of E- and Z-olefin 29 (66 mg, 51% yield, E/Z=89:11) as a colorless oil. The E/Z ratio of the crude products was determined by ¹H NMR in CDCl₃. Integration of the signal due to vinyl protons of the E-olefin (δH 6.22 ppm) versus that of Z-olefin (δH 5.65 ppm) gave the crude E/Z ratio as 89:11. E-olefin: ¹H NMR (400 MHz, CDCl₃) δ 7.34 (d, J=7.4 Hz, 2H; Ph-H), 7.28 (t, J=7.6 Hz, 2H; Ph-H), 7.18 (t, J=7.2 Hz, 1H; Ph-H), 6.37 (d, J=15.8 Hz, 1H; olefin-H), 6.22 (dt, J=15.8, 6.8 Hz, 1H), 2.20 (q, J=7.1 Hz, 2H), 1.50-1.43 (m, 2H), 1.30-1.26 (m, 20H), 0.88 (t, J=6.7 Hz, 3H); ¹³C NMR (100 MHz, CDCl₃) δ 138.0 (C), 131.3 (CH), 129.7 (CH), 128.5 (CH×2), 126.8 (CH), 125.9 (CH×2), 33.1 (CH₂), 32.0 (CH₂), 29.8 (CH₂x 3), 29.7 (CH₂×3), 29.6 (CH₂), 29.5 (CH₂), 29.4 (CH₂), 29.3 (CH₂), 22.8 (CH₂), 14.2 (CH₃); HRMS (APCI): m/z calculated for C₂₂H₃₅ ([M−H]+): 299.2733, found 299.2749. Z-olefin: ¹H NMR (400 MHz, CDCl₃) δ 7.24-7.14 (m, 5H, Ph-H), 6.38 (d, J=11.6 Hz, 1H; olefin-H), 5.65 (dt, J=11.6, 7.2 Hz, 1H), 2.31 (q, J=7.2 Hz, 2H), 1.46-1.39 (m, 2H), 1.28-1.24 (m, 20H), 0.86 (t, J=7.0 Hz, 3H); ¹³C NMR (100 MHz, CDCl₃) δ137.8 (C), 133.3 (CH), 128.7 (CH×2), 128.6 (CH), 128.1 (CH×2), 126.4 (CH), 31.9 (CH₂), 30.0 (CH₂), 29.7 (CH₂×3), 29.6 (CH₂×3), 29.5 (CH₂), 29.4 (CH₂), 29.2 (CH₂), 28.6 (CH₂), 22.7 (CH₂), 14.1 (CH₃); HRMS (APCI): m/z calculated for C₂₂H₃₅ ([M−H]+): 299.2733, found 299.2749.

### Example 12

### Differences in Hexadecane Utilization by Strains Rhodococcus sp. 008 and KSM-3BM

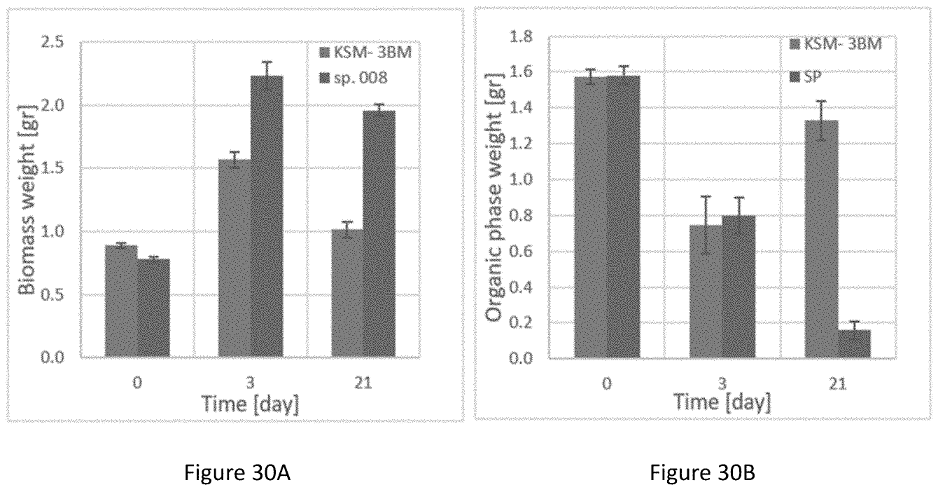

To characterize phenotypic differences in hexadecane utilization between the two strains, strains were grown on hexadecane and the accumulated biomass, organic phase leftovers and the appearance of hexadecene was tested in different time points. SP. 008 did not produce of hexadecene along the entire experiment (t=21 days), validating this strain has no alkane dehydrogenation activity and therefore is suitable to serve as a control. SP. 008 strain utilized the hexadecane to a near complete extent after 21 days, and produced significantly more biomass compared to KSM-3BM (FIG. 30), indicating that the utilization process of hexadecane by strain SP. 008 is more genetically efficient than that conducted by strain KSM-3BM.

### Example 13

### Phenotypic, Transcriptomic and Proteomic Analyses of KSM-3BM and SP 008 Grown on Dodecane and Hexadecane

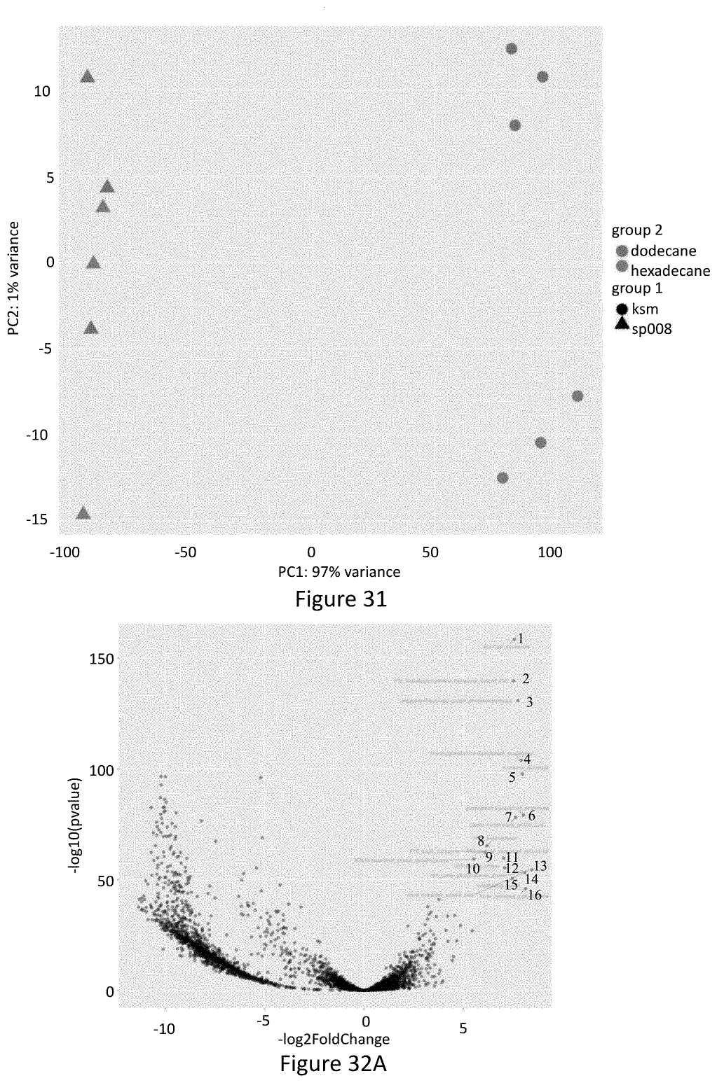

Comparative analysis of RNAseq data obtained by the two strains on two substrates, hexadecane and dodecane, showed big strain effect as compared to the substrate effect. More than 90% of the variance in the data can be explained by differences between strains while only 1% of the variance can be explained by the different substrates (FIG. 31). The substrate effect was observed only in KSM-3BM, in agreement with the phenotypic differences observed for this strain grown on hexadecane or dodecane.

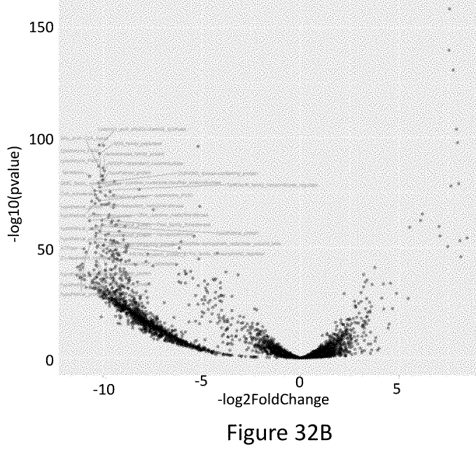

RNAseq data revealed 1,331 genes that showed different expression levels of at least ten-fold and adjusted p-value smaller than 0.05 between the two strains. 1,283 these genes were overexpressed by sp. 008 and may represent the outcome of better cell growth and division. In contrast, KSM showed overexpression of only 48 genes (Table 3) and may support the occurrence of only few main pathways and limited metabolic activity (FIG. 32). Some genes of this set were dramatically overexpressed, with acyl CoA desaturase being the most abundant gene (FIG. 32A). Moreover, two adjacent genes, ferredoxin reductase and hypothetical protein, are also highly expressed in KSM-3BM defining this highly expressed region as putative operon containing three genes: acyl-CoA desaturase, ferredoxin reductase and hypothetical protein, all highly expressed in ˜200 fold more in KSM-3BM as compared to sp 008. Proteomic analysis comparing KSM-3BM and sp. 008 grown on hexadecane demonstrated 200-7,000 proteins copies for these three genes. Importantly, ferredoxin reductase is important for the proper function acyl-CoA desaturase.

Proteomic results further demonstrated seven metabolic pathways that were significantly higher in SP compared to KSM-3BM (Table 2 below). The most significant pathway (Benjamini=2.6E-4) that include overexpression of ten genes is the beta-oxidation pathway for fatty acid degradation.

TABLE 2

| Category | Pathway | Term | Count | % | P-Value | Benjamini |
|---|---|---|---|---|---|---|
| KEGG | 1 | Fatty acid | 10 | 16.7 | 5.5E−6 | 2.6E−4 |
| pathway | | degradation | | | | |
| KEGG | 2 | Valine, Leucine | 10 | 16.7 | 1.4E−5 | 3.2E−4 |
| pathway | | and Isoleucine | | | | |
| | | degradation | | | | |
| UP_{——}Keywords | 3 | Plasmid | 13 | 21.7 | 2.3E−5 | 8.2E−4 |
| KEGG | 4 | Fatty acid | 9 | 15.0 | 8.0E−5 | 1.3E−3 |
| pathway | | metabolism | | | | |
| UP_Keywords | 5 | Acetyltransferase | 7 | 11.7 | 4.8E−4 | 8.5E−3 |
| KEGG | 6 | Propanoate | 7 | 11.7 | 1.5E−3 | 1.8E−2 |
| pathway | | metabolism | | | | |
| COG ontology | 7 | Lipid metabolism | 7 | 11.7 | 4.6E−3 | 4.5E−2 |

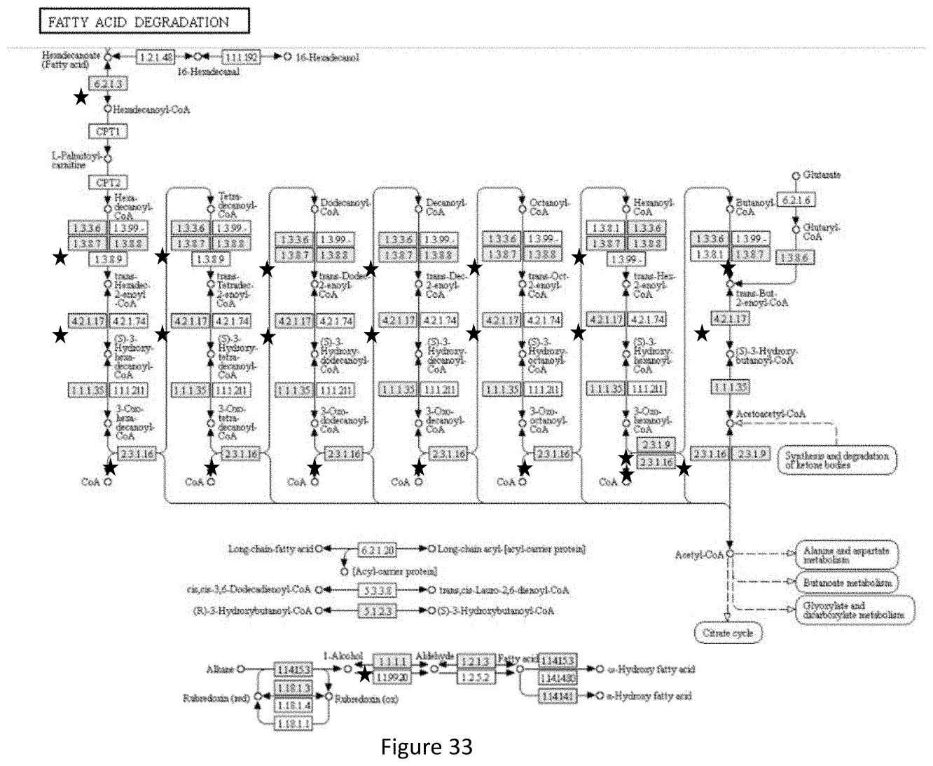

These results demonstrate silencing of the beta oxidation pathway in KSM-3BM (FIG. 33) while overexpressing the acyl CoA desaturase operon. Therefore, these results may raise the possible hypothesis that the overexpression of the acyl CoA desaturase in KSM-3BM results in nonspecific dehydrogenation that is conducted on the alkane (rather than on acyl CoA fatty acids, the known substrate of acyl CoA desaturase), as a main pathway for energy harvest, while no fatty acid oxidation occurs.

TABLE 3
Genes overexpressed in KSM

| Gene name | Gene product |
|---|---|
| WP 003945297.1 | Acyl-CoA desaturase |
| WP 065352723.1 | PTS N-acetylglucosamine transporter subunit IIBC |
| WP 065352722.1 | Phosphoenolpyruvate - protein phosphotransferase |
| WP 065352721.1 | N-acetylglucosamine-6-phosphate deacetylase |
| WP 050656813.1 | Ferredoxin reductase |
| WP 007726140.1 | Glucosamine-6-phosphate deaminase |
| WP 007726141.1 | Copper homeostasis protein CutC |
| WP 065351823.1 | HNH endonuclease |
| WP 003941324.1 | PTS sugar transporter |
| WP 019749215.1 | PTS glucose transporter subunit IIA |
| WP 003941638.1 | NDMA-dependent alcohol dehydrogenase |
| WP 042450649.1 | GTP-binding protein |
| WP 003941322.1 | HPr family phosphocarrier protein |
| WP 065351660.1 | Nitrile hydratase subunit beta |
| WP 003945295.1 | Hypothetical protein |
| WP 042450854.1 | Nitrile hydratase subunit alpha |
| WP 065351492.1 | LysR family transcriptional regulator |
| WP 003940783.1 | Flavodoxin family protein |
| WP 065351661.1 | Hypothetical protein |
| WP 065352563.1 | HNH endonuclease |
| WP 065351754.1 | HNH endonuclease |
| WP 030536487.1 | Rrf2 family transcriptional regulator |
| WP 065351402.1 | HNH endonuclease |
| WP 080726744.1 | Cytochrome P450 |
| WP 042449699.1 | TetR/AcrR family transcriptional regulator |
| WP 065351224.1 | Holo-ACP synthase |
| WP 003941275.1 | Methylmalonate-semialdehyde dehydrogenase (CoA |
| | acylating) |
| WP 003939932.1 | IclR family transcriptional regulator |
| WP 007729732.1 | GGDEF domain-containing protein |
| WP 003946304.1 | DUF1839 domain-containing protein |
| WP 042452232.1 | Hypothetical protein |
| WP 007726566.1 | Hypothetical protein |
| WP 054187331.1 | Acyl-CoA dehydrogenase |
| WP 003941308.1 | 3-hydroxyisobutyrate dehydrogenase |
| WP 007726568.1 | Hypothetical protein |
| WP 058227688.1 | Enoyl-CoA hydratase/isomerase family protein |
| WP 065351782.1 | Hypothetical protein |
| WP 065352072.1 | Hypothetical protein |
| WP 020968150.1 | Hypothetical protein |
| WP 003941552.1 | Heparin-binding hemagglutinin |
| WP 065352440.1 | Urea carboxylase |
| WP 003942314.1 | Nitrite reductase large subunit |
| WP 042450108.1 | NarK/NasA family nitrate transporter |
| WP 007735391.1 | Urea ABC transporter substrate-binding protein |
| WP 065352885.1 | Reductase |
| WP 007727244.1 | Purine permease |
| WP 065352214.1 | Isopenicillin N synthase family oxygenase |
| WP 003943732.1 | Twin-arginine translocase TatA/TatE family subunit |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for selectively functionalizing alkanes, comprising:
(a) providing an alkane;
(b) dehydrogenating at least one saturated hydrocarbon of said alkane by a hydrocarbon-utilizing microorganism; followed by
(c) isomerization-hydrofunctionalization reaction;
thereby yielding a functionalized alkane or derivative thereof.

2. The method of claim 1, wherein said alkane comprises at least one terminal C—H bond, wherein said alkane is selectively functionalized at one of the terminal C—H bonds at a yield of at least 98%.

3. The method of claim 1, wherein said hydrocarbon-utilizing microorganism is selected from the group consisting of a yeast, a cyanobacterium, and a bacterium.

4. The method of claim 3, wherein said hydrocarbon-utilizing microorganism is characterized by having overexpression of at least one gene selected from the group consisting of: WP_003945297.1; WP_065352723.1; WP_065352722.1; WP_065352721.1; WP_050656813.1; WP_007726140.1; WP_007726141.1; WP_065351823.1; WP_003941324.1; WP_019749215.1; WP_003941638.1; WP_042450649.1; WP_003941322.1; WP_065351660.1; WP_003945295.1; WP_042450854.1; WP_065351492.1; WP_003940783.1; WP_065351661.1; WP_065352563.1; WP_065351754.1; WP_030536487.1; WP_065351402.1; WP_080726744.1; WP_042449699.1; WP_065351224.1; WP_003941275.1; WP_003939932.1; WP_007729732.1; WP_003946304.1; WP_042452232.1; WP_007726566.1; WP_054187331.1; WP_003941308.1; WP_007726568.1; WP_058227688.1; WP_065351782.1; WP_065352072.1; WP_020968150.1; WP_003941552.1; WP_065352440.1; WP_003942314.1; WP_042450108.1; WP_007735391.1; WP_065352885.1; WP_007727244.1; WP_065352214.1; and WP_003943732.1, compared to a control.

5. The method of claim 3, wherein said hydrocarbon-utilizing bacterium is Rhodococcus mutant strain KSM-B-3M.

6. The method of claim 1, wherein said alkane is a linear, branched or cyclic alkane.

7. The method of claim 1, wherein said alkane is a C4 to C40 alkane.

8. The method of claim 1, wherein said alkane further comprises a functional group, an aryl substituent, or a combination thereof.

9. The method of claim 6, wherein said functionalized alkane or derivative thereof has a general formula RCₙH₂ₙR¹, wherein n is an integer having a value of 4 to 40, and wherein R, R¹ are each independently selected from a hydrogen atom, a halogen atom, a nitro group, an amine group, an azido group, a cyano group, a methoxy group, a carboxylic group, an ester group, an ether group, an aromatic group, alkyl group, vinyl group, an alcohol group, a carbamate group, an urea group or a combination thereof.

10. The method of claim 9, wherein said alkane is a C13 to C20 alkane.

11. The method of claim 10, wherein n has a value of 13 to 20.

12. The method of claim 11, wherein the yield of step (b) is at least 18%.

13. The method of claim 1, wherein step (b) is performed in any one of: (i) an aqueous medium selected from the group consisting of phosphate buffer, an amino acid, thiamine hydrochloride and magnesium sulfate heptahydrate, or any combination thereof, (ii) at a temperature ranging from 20° C. to 34° C., and (iii) for at least 5 days.

14. (canceled)

15. The method of claim 1, wherein the pH of the aqueous medium is within the range of 6.0 to 6.8.

16. (canceled)

17. The method of claim 1, wherein step (c) is metal assisted isomerization-hydrofunctionalization reaction.

18. The method of claim 17, wherein said isomerization-hydrofunctionalization reaction is selected from the group of halogenolysis, oxidation, copper-catalyzed allylation, hydroboration, hydrosilylation, hydrozirconation, hydroarylation and hydroamination.

19. The method of claim 1, wherein step (c) functionalizes said alkane with a terminal covalent bond selected from: C—C, C—O, C—X, wherein X is halogen, C—Si, C—N, C—B, C—P, C—Se, C—Zn, or any combination thereof.

20. The method of claim 1, wherein step (b) further comprises:
(i) recovering the microorganism; and
(ii) re-dissolving the microorganism in aqueous medium and performing another dehydrogenation reaction.

21. A composition comprising at least two cis-alkene regioisomers and an alkane wherein the ratio of said at least two cis-alkenes to said alkane is in a range of 95:5 to 80:20.

22. The composition of claim 21, wherein said alkane is hexadecane and said cis-alkene regioisomers are about 80% cis-7-hexadecene and about 20% cis-8-hexadecene, and wherein the ratio of said at least two cis-alkenes to said alkane is in a range of 90:10 to 80:20.
